SEQUENCE LISTING (15 sequences)

SEQ ID NO: 1 - DNA, 482 bp, Artificial Sequence
  CARD-CRE3
cattttattaaacactctaaaccagatccagtactaagtgtttcatatgaatttttttcatctaaccatcaacaaacttttaaggtaagcactatcatcctctcctcttacagatgaggatataaagcttagaaaaacaatgtgcaaaaatgctactcattaagcacagatgtgaaaaggaaaaagaaaatgccctgaaggcagctcgcatgagtataatgtctgagaagtttgttgataattgacaaagaatgaagttttctcaatcacaagccttgtagaaattgtggcgtcaagttgtgtcactgtcaataccaggtgattagtcaagtgattacataatggtctccctcagaaccaaaggtatgggcaagacaactggctaaagaattttgtgggtgacttctttcacatgagattaatttatcaaaagatgtcacacaggactctgccaactgacacgctaccagtttccatctgtt

SEQ ID NO: 2 - DNA, 355 bp, Artificial Sequence
  CARD-CRE8
cttatctccctccactgctgctacttaatttgtttcctctctgccacccctcaccagcatgtcagacatacaaaacaagggatttttgtgtgcttggcacacagtagatgcacaataaatgttgaagggctgaactaatttgggtttgagtcatagggagcttgggggatgtgggtgattggatagattctggagactttaggggactgggccgggggaaatgcggcctctaagctctccgctgaggcggcttggaaggaatagtgactgacgtggaggtgggggaggtggctggcccgggcgaggcccagggagagggagaggaggcgggtgggagaggaggagggtgtatctc

SEQ ID NO: 3 - DNA, 1640 bp, Artificial Sequence
  CARD-CRE11
aactgagttgtgtgacattcacgtggccccttcagttagcaccgcacggttggttaagaggagggttttattcaggcataaatattttgaaatatttgtaatcaaatcccacgtagtatagacaacctcccagatggtggaggggaaacacaccacccccctgactgggagagaaaagaccgcggttttctttcctgccacttgtcctcctccgggagcctcggtctcctctctgtgggacagggcagcctcccctggctcctcctggtgcaggatcgatgaggctcttgcccacagcctttgtcccccgacttcctaccacctggtgtcctcaccccatctgggccccgagcacacagcctctggtcctggtttccactgaaaatgtaaactatggtccacgccacattctccagtactcctgtgctattgtgaaagtaaaagacaaaaatacacgtgcttggcagcccgagagcctgggtgagattattgagaataatgcggtggctctggcttaaaaaataaaagccggaggatctaccaccagctctgaacacacacccgtgacacaaactgactcatgtgcaattgcccattagtggcaggtcttgccatttttcgtttttttctgagtcgagatcagctgcggctggggagaacattagttctgtctgtcggctccatgagtaagcagaggaaagtctgactacagaggcatcttctcaaatgtccatgaaaggctgcaagcccgagagtaacgcccctgctgccctgagtggtccacgtggtgtccagagtgtgagaaagccacagagagcttttccacacggagccacttaaggcctgcctcagcctgagaacacttgggcagaaaaggacagcacggcttcctcctcctccaggccgggcctacaagcacccacagttccgacagagaaagtctcttcttaatggtacgcatgtgtcttactgtagctcccagttagtcccctaagcgtgaaccaccacccacccatgctgctcccctcccaccatctctccataaacaggacccaacgcagccaaaaactcatggtcatgtgaccttggcatcagcttgctcgttaggaaccgtgcctggaggtgctctgagcactgactgccaacctcagcagggccctttgtggtgttgaggtgtggtgtccgcagggcagcctgcagcctgctggggtccagcctggggcctcaggtcctgggaaccctggttgaggctgggctgcctcagtgcccctccgcagcctgctgcacccaagcactctgagaaaaggagcaatcacccaacttcagtctgaaaatgagaatgttgagaagggcatgatgaggggaagcagaccagagcccaccggcaagggtagaacgtcagccccgccctagtgggacagacaagagagaagcctcctcacaccaggaggcagtatagccggagccttccctccagggctctcaggagagcctgtgctggttcatgaaaaagtgaggcctctgggctttgctgctgcagagtgaggtcagagcccacaggccccacagatcatcctgcctgacaagcacaggaggcaccactgcttgaattcagagggcagctccgagggtcctaggtc

SEQ ID NO: 4 - DNA, 495 bp, Artificial Sequence
  CARD-CRE12
gacattcacgtggccccttcagttagcaccgcacggttggttaagaggagggttttattcaggcataaatattttgaaatatttgtaatcaaatcccacgtagtatagacaacctcccagatggtggaggggaaacacaccacccccctgactgggagagaaaagaccgcggttttctttcctgccacttgtcctcctccgggagcctcggtctcctctctgtgggacagggcagcctcccctggctcctcctggtgcaggatcgatgaggctcttgcccacagcctttgtcccccgacttcctaccacctggtgtcctcaccccatctgggccccgagcacacagcctctggtcctggtttccactgaaaatgtaaactatggtccacgccacattctccagtactcctgtgctattgtgaaagtaaaagacaaaaatacacgtgcttggcagcccgagagcctgggtgagattattgagaataatgcggtggc

SEQ ID NO: 5 - DNA, 548 bp, Artificial Sequence
  CARD-CRE14
ccccaccccaccccttacttgtttttccttctactcattttctcctccctccctctcccctctccccacactagaaccatcagggccctcaccagggcattcagttcaggttctgggtcagctctcacagctctaccagtcccacccccatccccaggaaaaactgctccttatttggagtcacaaaaatatttaacagagatctaactgaccactaaaaattcctcctttaaaacaaacacctaatcaactattttcccccaagttatatggaaaaacagctgcaattagaacttgattctcactttaagaaagaaagattcttgtttggttttctccactttcattttttgtttctaggtccagggcctcccaccaaatgctgacggctgcctgcttcaaaccctgccacatcagcagggagggagcacagcggcatttggtatttgcatcagtttccaggaatgcttttcaagttatcagttcactctgctgcctttagcagagacgttttccctctaagtttatagatgtctgcttcaatttac

SEQ ID NO: 6 - DNA, 844 bp, Artificial Sequence
  CARD-CRE16
agctgagatcacgccactgcactccagcctgggcgacagagcgagactccatctcaaaaataaatatataaataaataaaaataaaaaagagaaaggacagggtgcacagagccaggaccccaccccctcctggtctggaccaccctgggccatctcctaagcttgacactctcaggcctgaggctgtcaggccagggcgctttgactatcccctgcctgcccctgaacgcggccaagcccgaggccttagccaagccctgcagcctcaggcttggcgcccggcccagcctttctttagccttcccgaggagccagcgagagagcgctgcagcagccgcagtccttgcttccggggaaggcccgggtggggccagggcctgtggttaagtcagtggccctctgcagacgcgcacgcagcaggagccacgccctggtccctgcccgggagtggcctggtctcccccagctctgattccctgaaggaccctgcacctcctcagccatcttctctgggcaccccctgagcccagcctgctggccacaggccccttcccgccaacttgtgggcctagctcatcatcaaactattttccactggcttcccccaatctctgccacagcacaattttggtggggaggtggagagggatgaaggagaataggaggtggagcttggactctggccgggggcggcggggtgagggggggcagcagctcgccactctgattggtcacctctgctccaaaactggcttcaaaattccacggactccgcccccagtggcccccagccctatcctgacttgcaattggctgaactttcagggggcggggctcacccgg

SEQ ID NO: 7 - DNA, 300 bp, Artificial Sequence
  CARD-CRE17
ctgctggccacaggccccttcccgccaacttgtgggcctagctcatcatcaaactattttccactggcttcccccaatctctgccacagcacaattttggtggggaggtggagagggatgaaggagaataggaggtggagcttggactctggccgggggcggcggggtgagggggggcagcagctcgccactctgattggtcacctctgctccaaaactggcttcaaaattccacggactccgcccccagtggcccccagccctatcctgacttgcaattggctgaactttcagggggcg

SEQ ID NO: 8 - DNA, 1478 bp, Artificial Sequence
  CARD-CRE20
aagggcgcgagtgagccgtgtcgggccggccggcgccttaaacccgccgccccgcccggcctgtccgcgggaaccacaccccggggcggtgagagcacgggggccccgcccgtgccgccctcccggccccgcggggcctgcgcgcgcgctcctgctggcgtcggggggtgagcgggcacccggcgtgaccggcccatgggcgctcggtctcacgcccgcttcgcagagctcaccccgccagctcctgctcggtccccagtcccctgccccgagccctcagggatcgcaccgcagtgcgagcggctcttccaccctctggtttctccccgtttccggtcaatatatttaaacttcttaacaaccacggcttcgagacgatccagggtttcatctattaaatcctccgtgggcctgagacacctgagtgctcattactctgtggtttgggaagctctcttaagaacaagctgttaaaaacccccacttgtggactgtgtccccgctggccacaggcctgctgtggtcttgggtgtcaagcactaaaaggacaaaccgttgccaaccacttccctttctggaacctcttaactgcctgcacctccctccccgctttgtctcccagtggcattcaacaagactctgtgctttttttgtgctgagccgttttcagtgagtactggaatccgtgtgagagctgtgcccttgttttgagttcttgctgaactgctcgccctccagcaagcctctcgggagaacagagcgcagctgagaggagcaggggtcccagctccttcctcgttggagctgccacggaggcacctaaagcgcccccagtcttcaccagcattccaggaggtttttgatataaacggtggactcttcctcagtctttctgcgaagtgctctgcaaactcaacttgaaagcctctcttcccaggaacactggcctctccaatgcagacatggaagttgctttcactttttaactgaatgtgtaacattgcagcgtgcattattctgcatgtcacattgtggttcttttgtgttcttttttcttagcataaatccagggatttacagggttgctgactgaaagccagcagttcagttcataatcccattagcagtgtaaagaactcattctcaacagactcttacaaggataattattgcaaattttttgaaaattaaatgaccacaaaatcggggggtggagagtggggaaacatcctccaatccagaaaaacctagcactgctttttggcctctccttagtgtattatagacaagtggagcattttttttcccctcaaaggtcatatgttgacctgacagttgtttaaaaaacaatgaaaaatctttacagcagagtttccctaaatatttttgtctttgttccatcctaagagatgcagtttacatgacccatttgttttcttgaaaaggaaacaaatgcagagtgcactctatttcctatt

SEQ ID NO: 9 - DNA, 20 bp, Artificial Sequence
  Luciferase forward primer
cccaccgtcgtattcgtgag

SEQ ID NO: 10 - DNA, 21 bp, Artificial Sequence
  Luciferase reverse primer
tcagggcgatggttttgtccc

SEQ ID NO: 11 - DNA, 334 bp, Artificial Sequence
  SPc5-12 promoter
tggccaccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctaga

SEQ ID NO: 12 - DNA, 92 bp, Artificial Sequence
  MVM intron
aagaggtaagggtttaagggatggttggttggtggggtattaatgtttaattacctggagcacctgcctgaaatcactttttttcaggttgg

SEQ ID NO: 13 - DNA, 49 bp, Artificial Sequence
  pA signal
aataaaagatctttattttcattagatctgtgtgttggttttttgtgtg

SEQ ID NO: 14 - DNA, 567 bp, Artificial Sequence
  hMLC promoter
atgagccacagtgccagccttcatggttattttaaagatggtggtcggggaggcttcactcaggagatgacatatgagcaaagatgcagtgaaggaggtgaaggaaggagccgtgcgatgactgacagaaagacattccaggtagagggcacacaggtgcaaagaccctgaggccagatccaggctgataaaacagagcattttagcagtctcctctccctgccattttttttctcaaaattgacaagcacaagtgtccccggcccaagcaccgcagagagcgcgcagcatctctccccgtgaccatgacccagctactgcctctttaaccttgaatgcctttttgggggctcacgtgtcacccagtggcgagtgagccacccttacttcagaagaacggcatggggtgggggggccttaggtggtgcccgcctcacctatgactgccaaaagcggtcatggggttatttttaaacatggggaggaagtatttattgttcctgggctgcagagagctgggcggagtgtggaattcttctcgggaggcagtgctgggtcctttccacc

SEQ ID NO: 15 - DNA, 663 bp, Artificial Sequence
  CMV promoter
acgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcc

(19) United States
(12) Patent Application Publication
(10) Pub. No.: US 2024/0050591 A1
(43) Pub. Date: Feb. 15, 2024

(54) CARDIOMYOCYTE-DERIVED NUCLEIC ACID REGULATORY ELEMENTS AND METHODS AND USE THEREOF

(71) Applicant: VRIJE UNIVERSITEIT BRUSSEL, Brussel (BE)

(72) Inventors: Lay Khim Chuah, Bierbeek (BE); Thierry Vandendriessche, Bierbeek (BE)

(21) Appl. No.: 18/258,495

(22) PCT Filed: Dec. 21, 2021
(86) PCT No.: PCT/EP2021/087020

§ 371 Date: Jun. 20, 2023

(30) Foreign Application Priority Data
Dec. 21, 2020 (EP) 20216054.5

(51) Int. Cl. A61K 48/00; C12N 15/86; C12N 15/113

(52) U.S. Cl. CPC A61K 48/0058; C12N 15/86; C12N 15/113; C12N 2830/85; C12N 2310/141; C12N 2310/532

(57) ABSTRACT

The present invention relates to nucleic acid regulatory elements that are able to enhance heart-and/or muscle-targeted expression of genes, in particular heart- and muscle-targeted gene expression, more particularly gene expression in cardiomyocytes, methods employing these regulatory elements and uses of these elements. Expression cassettes and vectors containing these nucleic acid regulatory elements are also disclosed. The present invention is particularly useful for applications using gene therapy, more particularly heart- and/or muscle-directed gene therapy, e.g. for the treatment of cardiovascular diseases and disorders and muscle disorders, as well as other diseases and disorders that may benefit from high transgene expression in heart and/or muscle cells or tissue, and for vaccination purposes.

### CROSS REFERENCE TO RELATED APPLICATIONS

This application is a national phase entry under 35 U.S.C. § 371 of International Patent Application PCT/EP2021/087020, filed Dec. 21, 2021, designating the United States of America and published in English as International Patent Publication WO 2022/136388 on Jun. 30, 2022, which claims the benefit under Article 8 of the Patent Cooperation Treaty to European Patent Application Serial No. 20216054.5, filed Dec. 21, 2020, the entireties of which are hereby incorporated by

### REFERENCE

### Field

The present invention relates to nucleic acid regulatory elements that are able to specifically enhance gene expression in heart and/or muscle cells or tissue, in particular in heart and muscle cells or tissue, more particularly in cardiomyocytes, methods employing these regulatory elements and uses thereof. The invention further encompasses expression cassettes, vectors and pharmaceutical compositions comprising these regulatory elements. The present invention is particularly useful for the treatment of cardiovascular diseases and disorders, in particular coronary heart disease and heart failure, and muscle disorders and diseases, using gene therapy, or for the treatment of diseases that require secretion of therapeutic proteins from heart or muscle, or for vaccination purposes.

### BACKGROUND

Coronary heart disease (CHD) is the most common type of heart disease due to build-up of plaque in the heart arteries that could lead to a sudden heart attack (myocardial infarction) or to chronic ischemic cardiomyopathy. Heart failure (HF) is a most common consequence of CHD that develops when the heart muscle cannot pump sufficiently to meet the body's needs for blood and oxygen. Despite remarkable progress made by surgical and medical therapies, including resynchronization therapy and use of ventricular assist devices (Birks. 2013. Circulation Research 113:777-791), the long-term survival of patients with HF remains poor. Hence, there is a need for effective and safe treatments for CHD and HF.

Non-coding RNAs (ncRNAs) such as microRNAs (miRNA) and long non-coding RNAs (lncRNA), as well as circular RNAs (circRNA), have been identified as critical novel regulators of cellular processes, and expression of these non-coding molecules seems to be strictly regulated in physiological conditions as well as in several human diseases (reviewed in Beermann et al. 2016 Physiol Rev 96:1297-1325), including cardiovascular diseases (reviewed in Poller et al. 2018 Eur Heart J. 39:2704-2716) and/or muscle disorders. They are rapidly emerging as fundamentally novel therapeutics and attractive alternatives to protein-based approaches. Modulating ncRNAs (e.g. by over-expression or inhibition) has unprecedented potential to activate or de-activate specific genetic programs, leading to the development of innovative genetic medicines. In particular, these emerging insights paved the way towards the use of ncRNA-based gene therapy as a novel treatment modality for cardiovascular diseases (CVD) and/or muscle disorders. There are several advantages of ncRNAs (or their cognate inhibitors) as a therapeutic modality compared to more conventional protein-based approaches. Typically, ncRNA can hit more than one pathway resulting in potential additive or synergistic effects. Some ncRNA may even be equivalent to a ‘molecular master-switch’ critically important in regulating cellular physiology in normal or pathologic conditions, particularly in the context of CHD and HF. As some ncRNAs (lncRNAs) naturally function at relatively low copy number per cell (less than 100), therapeutic efficacy can be achieved with lower amounts of vectors. Some specific ncRNAs are highly promising targets and have been shown to have beneficial effects in animal models of diseases, including CHD and HF, justifying their exploration in clinical trials.

Additionally, there are many hereditary disorders that are due to a gene defect that impair the function of skeletal muscle and heart (e.g. Pompe disease, muscular dystrophies etc.). This defect may ultimately result in severe muscle weakness and paralysis or cardiopulmonary failure with life-threatening consequences.

It is an aim of the present invention to develop an innovative gene therapy platform to treat cardiovascular diseases, in particular coronary heart disease (CHD) and heart failure (HF), and muscle-related disorders. Furthermore, maximizing gene expression in muscle also has implications for gene vaccination and could impact on non-muscle diseases that would benefit from increased expression of circulating therapeutic proteins (e.g. hemophilia).

However, gene therapy to muscle and/or heart cells or tissue is relatively inefficient due to limitations in gene delivery and gene expression. Conventional vector designs based on standard heart and/or muscle-targeting promoters typically result in sub-optimal expression in the desired target tissue. Cis-regulatory elements (CRE) (also referred to as cis-regulatory modules (CRM)) have initially been identified in whole tissues/organs, which comprise different cell types. These CREs allow to boost gene expression in heart, muscle and/or diaphragm (Sarcar et al. 2019. Nat Commun. 10(1):492; WO 2015/110449; WO 2018/178067; WO 2011/051450). These particular CREs were previously identified using the differential distance matrix (DDM)/multidimensional scaling algorithm (MDS) of De Bleser et al. (2007. Genome Biol 8, R83), which relied on the identification of arrays of transcription factor binding sites (TFBS) that were common among genes that are highly expressed in a given tissue/organ. Consequently, genes that are highly expressed in the tissue/organ, but which do not share such an array of common TFBS were filtered out by this bioinformatics algorithm.

There remains however a need in the art for safe and efficient gene therapy and more particularly for safe and efficient expression of the gene of interest in heart and/or muscle cells or tissue, in particular in cardiomyocytes.

### SUMMARY

The present invention addresses the need for efficient gene therapy for cardiovascular diseases, in particular coronary heart disease and heart failure, and muscle disorders, as well as other diseases and disorders that may benefit from high transgene expression in heart and/or muscle cells or tissue. An approach was developed to maximize (trans)gene expression in heart, in particular in heart muscle cells or tissue, and other types of muscle cells or tissue, based on cardiomyocyte-derived cis-regulatory modules (CARD-CREs), which confer high (trans)gene expression in cardiomyocytes, but also in skeletal muscle and diaphragm cells or tissue. The use of a CARD-CRE element identified herein enhanced (trans)gene expression in the heart and various skeletal muscle and diaphragm cells or tissue compared to (trans)gene expression from the promoter alone. Hence, the use of these novel CARD-CREs constitutes an attractive strategy to maximize the overall efficacy and safety of gene therapy for cardiovascular diseases, in particular coronary heart disease and heart failure, and muscle disorders as well as other diseases and disorders that may benefit from high transgene expression in heart and/or muscle cells or tissue. In addition, lower vector doses may be required to achieve the same or even improved therapeutic effects.

The invention therefore provides the following aspects:

Aspect 1: an isolated nucleic acid regulatory element for enhancing heart- and/or muscle-targeted gene expression, preferably heart- and muscle-targeted gene expression, comprising, consisting essentially of, or consisting of a sequence selected from the group consisting of: SEQ ID NO:2, SEQ ID NO:3, SEQ ID NO:5, SEQ ID NO:6, SEQ ID NO:7 and SEQ ID NO:8, a sequence having at least 80%, preferably at least 85%, more preferably at least 90%, even more preferably at least 95%, such as 95%, 96%, 97%, 98%, or 99%, identity to (the full-length of) any one of these sequences (i.e. a sequence that has at least 80%, preferably at least 85%, more preferably at least 90%, even more preferably at least 95%, such as 95%, 96%, 97%, 98%, or 99%, identity to (the full-length of) the sequence set forth in SEQ ID NO:2, SEQ ID NO:3, SEQ ID NO:5, SEQ ID NO:6, SEQ ID NO:7 or SEQ ID NO:8), or a functional fragment thereof. In preferred embodiments of said aspect, a nucleic acid regulatory element is maximal 2000 nucleotides, preferably maximal 1900 or 1800 nucleotides, more preferably maximal 1700 nucleotides long and comprises, consists essentially of, or consists of a sequence selected from the group consisting of: SEQ ID NO:2, SEQ ID NO:3, SEQ ID NO:5, SEQ ID NO:6, SEQ ID NO:7 and SEQ ID NO:8, a sequence having at least 80%, preferably at least 85%, more preferably at least 90%, even more preferably at least 95%, such as 95%, 96%, 97%, 98%, or 99%, identity to (the full-length of) any one of these sequences, or a functional fragment thereof.

In embodiments of said aspect, said nucleic acid regulatory element is maximal 1000 nucleotides long, preferably maximal 900, 800, 700, 600 or 500 nucleotides, more preferably maximal 400 nucleotides, and comprises, consists essentially of or consists of the sequence set forth in SEQ ID NO:2, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:2, or a functional fragment thereof. In a particular embodiment of said aspect, said regulatory element consists of the sequence set forth in SEQ ID NO:2 (i.e. is CARD-CRE8). In embodiments of said aspect, said nucleic acid regulatory element is maximal 2000 nucleotides, preferably maximal 1900 or 1800 nucleotides, more preferably maximal 1700 nucleotides long and comprises, consists essentially of or consists of the sequence set forth in SEQ ID NO:3, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:3, or a functional fragment thereof. In a particular embodiment of said aspect, said regulatory element consists of the sequence set forth in SEQ ID NO:3 (i.e. is CARD-CRE11). In embodiments of said aspect, said nucleic acid regulatory element is maximal 1000 nucleotides long, preferably maximal 900, 800 or 700 nucleotides, more preferably maximal 600 nucleotides, and comprises, consists essentially of or consists of the sequence set forth in SEQ ID NO:5, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:5, or a functional fragment thereof. In a particular embodiment of said aspect, said regulatory element consists of the sequence set forth in SEQ ID NO:5 (i.e. is CARD-CRE14). In embodiments of said aspect, said nucleic acid regulatory element is maximal 1500 nucleotides long, preferably maximal 1400, 1300, 1200, 1100 or 1000 nucleotides, more preferably maximal 900 nucleotides, and comprises, consists essentially of or consists of the sequence set forth in SEQ ID NO:6, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:6, or a functional fragment thereof. In a particular embodiment of said aspect, said regulatory element consists of the sequence set forth in SEQ ID NO:6 (i.e. is CARD-CRE16), or a functional fragment thereof. In embodiments of said aspect, said nucleic acid regulatory element is maximal 1000 nucleotides long, preferably maximal 900, 800, 700, 600 or 500 nucleotides, more preferably maximal 450 or 400 nucleotides, and comprises, consists essentially of or consists of the sequence set forth in SEQ ID NO:7, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:7, or a functional fragment thereof. In a particular embodiment of said aspect, said regulatory element consists of the sequence set forth in SEQ ID NO:7 (i.e. is CARD-CRE17). In embodiments of said aspect, said nucleic acid regulatory element is maximal 1700 nucleotides long, preferably maximal 1600 nucleotides, more preferably maximal 1500 nucleotides, and comprises, consists essentially of or consists of the sequence set forth in SEQ ID NO:8, a sequence having at least 80%, preferably at least 85%, more preferably at least 90%, even more preferably at least 95%, such as 95%, 96%, 97%, 98%, or 99%, identity to (the full-length of) the sequence set forth in SEQ ID NO:8, or a functional fragment thereof. In a particular embodiment of said aspect, said regulatory element consists of the sequence set forth in SEQ ID NO:8 (i.e. is CARD-CRE20).

Aspect 2: the nucleic acid regulatory element according to aspect 1, comprising, consisting essentially of or consisting of a functional fragment of a sequence selected from the group consisting of: SEQ ID NO:2, SEQ ID NO:3, SEQ ID NO:5, SEQ ID NO:6, SEQ ID NO:7, SEQ ID NO:8, wherein said functional fragment comprises, consists essentially of or consists of at least 20, preferably at least 25, more preferably at least 50, at least 100, at least 200 or at least 250, contiguous nucleotides from the sequence from which it is derived.

In embodiments of said aspect, said nucleic acid regulatory element is a functional fragment of SEQ ID NO: 2, wherein said functional fragment comprises or consists of at least 310, preferably at least 320, 330 or 340, more preferably at least 345 or 350 contiguous nucleotides from SEQ ID NO:2. In embodiments of said aspect, said nucleic acid regulatory element is a functional fragment of SEQ ID NO: 3, wherein said functional fragment comprises or consists of at least 1600, preferably at least 1610 or 1620, more preferably at least 1630 or 1635 contiguous nucleotides from SEQ ID NO:3. In embodiments of said aspect, said nucleic acid regulatory element is a functional fragment of SEQ ID NO: 5, wherein said functional fragment comprises or consists of at least 500, preferably at least 510, 520 or 530, more preferably at least 535 or 540 contiguous nucleotides from SEQ ID NO:5. In embodiments of said aspect, said nucleic acid regulatory element is a functional fragment of SEQ ID NO: 6, wherein said functional fragment comprises or consists of at least 800, preferably at least 810 or 820, more preferably at least 830 or 835 contiguous nucleotides from SEQ ID NO:6. In embodiments of said aspect, said nucleic acid regulatory element is a functional fragment of SEQ ID NO: 7, wherein said functional fragment comprises or consists of at least 260, preferably at least 270 or 280, more preferably at least 290 or 295 contiguous nucleotides from SEQ ID NO:7. In embodiments of said aspect, said nucleic acid regulatory element is a functional fragment of SEQ ID NO: 8, wherein said functional fragment comprises or consists of at least 1435, preferably at least 1440, 1450 or 1460, more preferably at least 1465 or 1470 contiguous nucleotides from SEQ ID NO:8.

Aspect 3: a nucleic acid regulatory element for enhancing heart- and/or muscle-targeted gene expression, preferably heart- and muscle-targeted gene expression, comprising, consisting essentially of, or consisting of the complement of a sequence as defined in aspect 1 or 2.

Aspect 4: a nucleic acid regulatory element for enhancing heart- and/or muscle-targeted gene expression, preferably heart- and muscle-targeted gene expression, hybridizing under stringent conditions to the nucleic acid regulatory element according to any one of aspects 1 to 3.

Aspect 5: the nucleic acid regulatory element according to any one of aspects 1 to 4, having a total length of 1700 nucleotides or less, preferably 1500 nucleotides or less, more preferably 900 nucleotides or less, even more preferably 600 nucleotides or less.

Aspect 6: use, preferably an in vitro or ex vivo use, of the nucleic acid regulatory element according to any one aspects 1 to 5 in a nucleic acid expression cassette or a vector, in particular for enhancing gene expression in heart and/or muscle cells or tissue, more particularly in heart and muscle cells or tissue, even more particularly for enhancing gene expression in cardiomyocytes.

Aspect 7: a nucleic acid expression cassette comprising at least one, such as one, two, three, four, five or more, nucleic acid regulatory element according to any one of aspects 1 to 5, operably linked to a promoter.

Aspect 8: the nucleic acid expression cassette according to aspect 7, wherein the at least one nucleic acid regulatory element is operably linked to a promoter and a transgene.

In particular embodiments of this aspect, the transgene encodes acid α-glucosidase (GAA) (e.g. GAA as a secreted or native form). In other particular embodiments of this aspect, the transgene encodes a sarcoglycan, in particular a sarcoglycan selected from an alpha-sarcoglycan, a beta-sarcoglycan and a gamma-sarcoglycan, preferably a beta-sarcoglycan. In particular embodiments, the transgene encodes an antibody or a nanobody.

Aspect 9: the nucleic acid expression cassette according to aspect 7 or 8, wherein the promoter is a heart- and/or muscle-targeted promoter, preferably a heart- and muscle-targeted promoter.

Aspect 10: the nucleic acid expression cassette according any one of aspects 7 to 9, wherein the promoter is selected from the group consisting of the hMLC promoter, in particular the hMLC promoter defined by SEQ ID NO:14, the SPc5-12 promoter, in particular the SPc5-12 promoter defined by SEQ ID NO:11, the desmin (DES) promoter and the MHCK7 promoter, such as a promoter selected from the group consisting of the SPc5-12 promoter, in particular the SPc5-12 promoter defined by SEQ ID NO:11, the desmin (DES) promoter and the MHCK7 promoter.

The use of a chimeric SPc5-12 promoter that also incorporates a CARD-CRE element identified herein, enhances gene expression in heart and various skeletal muscle cells or tissue compared to the synthetic muscle-directed SPc5-12 promoter. Moreover, these novel CARD-CRE elements in conjunction with the SPc5-12 promoter yielded much higher gene expression than the CMV promoter, while preventing ectopic expression in non-target tissues such as the liver, contrary to the CMV promoter.

Aspect 11: the nucleic acid expression cassette according any one of aspects 7 or 8, wherein the promoter is an ubiquitously expressed promoter, preferably a promoter selected from the CMV promoter, in particular the CMV promoter as defined by SEQ ID NO:15, an RNA polymerase II promoter or an RNA polymerase III promoter, such as a promoter selected from an RNA polymerase II promoter or an RNA polymerase III promoter. In embodiments, the RNA polymerase III promoter is an U6 polymerase III promoter or a H1 polymerase III promoter.

Aspect 12: the nucleic acid expression cassette according to any one of aspects 8 to 10, wherein the transgene encodes a therapeutic protein or an immunogenic protein.

Aspect 13: the nucleic acid expression cassette according to any one of aspects 8 or 11, wherein the transgene encodes a non-coding RNA, preferably a microRNA, a long non-coding RNA (lncRNA), a circular RNA or a small interfering RNA (siRNA).

Aspect 14: the nucleic acid expression cassette according to any one of aspects 7 to 13, further comprising an intron, preferably a Minute Virus of Mouse (MVM) intron, more preferably the MVM intron defined by SEQ ID NO:12.

Aspect 15: the nucleic acid expression cassette according to any one of aspects 7 to 14, further comprising a polyadenylation signal, preferably a synthetic polyadenylation signal, more preferably the polyadenylation signal defined by SEQ ID NO:13.

Aspect 16: a vector comprising the nucleic acid regulatory element according to any one of aspects 1 to 5, or the nucleic acid expression cassette according to any one of aspects 7 to 15.

Aspect 17: the vector according to aspect 16, which is a viral vector, preferably an adeno-associated viral (AAV) vector, an adenoviral vector or a lentiviral vector.

Aspect 18: the vector according to aspect 16, which is a non-viral vector, preferably a plasmid, a minicircle or a transposon-based vector, such as a PiggyBac-based vector or a Sleeping Beauty-based vector.

Aspect 19: a pharmaceutical composition comprising the nucleic acid expression cassette according to any one of aspects 7 to 15, or the vector according to any one of aspects 16 to 18, and a pharmaceutically acceptable carrier.

Aspect 20: the nucleic acid regulatory element according to any one of aspects 1 to 5, the nucleic acid expression cassette according to any one of aspects 7 to 15, the vector according to any one of aspects 16 to 18, or the pharmaceutical composition according to aspect 19 for use in medicine.

Aspect 21: the nucleic acid regulatory element according to any one of aspects 1 to 5, the nucleic acid expression cassette according to any one of aspects 7 to 15, the vector according to any one of aspects 16 to 18, or the pharmaceutical composition according to aspect 19 for use in gene therapy, preferably heart and/or muscle (cell or tissue)-directed gene therapy.

In embodiments of this aspect, the nucleic acid expression cassette, the vector, or the pharmaceutical composition is for use in heart (cell or tissue)-directed gene therapy, in particular cardiomyocyte-directed gene therapy.

In embodiments, the nucleic acid expression cassette, the vector, or the pharmaceutical composition is for use in muscle (cell or tissue)-directed gene therapy.

Aspect 22: the nucleic acid expression cassette according to any one of aspects 7 to 15, or the vector according to any one of aspects 16 to 18, or a pharmaceutical composition comprising said nucleic acid expression cassette or said vector, for use in a method of producing antibodies or nanobodies in a subject, the method comprising introducing the nucleic acid expression cassette or the vector into heart and/or muscle cells of the subject, preferably into heart and muscle cells of the subject, more preferably into cardiomyocytes of the subject, in an amount effective to elicit expression of an antibody or a nanobody, wherein the transgene in the nucleic acid expression cassette or in the vector encodes the antibody or the nanobody.

In embodiments of this aspect, the subject is administered the nucleic acid expression cassette, the vector or the pharmaceutical composition.

In other embodiments of this aspect, the subject is administered the heart and/or muscle cells, preferably the heart and muscle cells, more preferably the cardiomyocytes, into which the nucleic acid expression cassette or the vector has been introduced.

Aspect 23: the nucleic acid regulatory element according to any one of aspects 1 to 5, the nucleic acid expression cassette according to any one of aspects 7 to 15, the vector according to any one of aspects 16 to 18, or the pharmaceutical composition according to aspect 19 for use in the treatment of a disease or disorder selected from the group comprising: cardiovascular diseases and disorders, lysosomal storage diseases, mitochondrial disorders (e.g. Barth syndrome), channelopathy (e.g. Brugada syndrome), metabolic disorders, myotubular myopathy (MTM), muscular dystrophy (e.g. Duchenne muscular dystrophy (DMD), Becker muscular dystrophy (BMD)), myotonic dystrophy, Myotonic Muscular Dystrophy (DM), Miyoshi myopathy, Fukuyama type congenital, dysferlinopathies, neuromuscular disease, motor neuron diseases (MND) (e.g. Charcot-Marie-Tooth disease (CMT), spinal muscular atrophy (SMA) or amyotrophic lateral sclerosis (ALS)), Emery-Dreifuss muscular dystrophy, facioscapulohumeral muscular dystrophy (FSHD), congenital muscular dystrophies, congenital myopathies, limb girdle muscular dystrophy (e.g. Limb Girdle Muscular Dystrophy type 2E (LGMD2E), Limb Girdle Muscular Dystrophy type 2D (LGMD2D), Limb Girdle Muscular Dystrophy type 2C (LGMD2C), Limb Girdle Muscular Dystrophy type 2B (LGMD2B), Limb Girdle Muscular Dystrophy type 2L (LGMD2L), Limb Girdle Muscular Dystrophy type 2A (LGMD2A)), metabolic myopathies, muscle inflammatory diseases, myasthenia, mitochondrial myopathies, anomalies of ionic channels, nuclear envelop diseases, distal myopathies, coagulation disorders (e.g. hemophilia A and B, FVII deficiency, von Willebrand's disease), C1 inhibitor deficiency or hereditary angioedema, diabetes, al-antitrypsin deficiency and kidney failure.

In embodiments of this aspect, the nucleic acid expression cassette, the vector, or the pharmaceutical composition is for use in the treatment of a cardiovascular disease or disorder, or a muscle disorder. In embodiments, the nucleic acid expression cassette, the vector, or the pharmaceutical composition is for use in the treatment of a cardiovascular disease or disorder. In further embodiments of this aspect, the disease or disorder is a cardiovascular disease or disorder selected from the group comprising: atherosclerosis, arteriosclerosis, coronary heart disease or coronary artery disease, peripheral arterial disease, congenital heart disease, congestive heart failure, heart failure or cardiac insufficiency, myocardial infarction or heart attack, cardiac ischemia, acute coronary syndrome or unstable angina and stable angina, cardiomyopathy (such as hypertrophic cardiomyopathy, dilated cardiomyopathy, restrictive cardiomyopathy and, primary cardiomyopathies caused by genetic mutations, such as Brugada syndrome and Fabry disease), cardiac amyloidosis (or “stiff heart syndrome”), myocarditis (or inflammatory cardiomyopathy), valvular heart disease, pericarditis, cardiac tamponade (also known as pericardial), endocarditis, cardiac arrhythmia (such as primary cardiac arrhythmias caused by genetic mutations, such as Brugada syndrome), hypertension, hypotension, vessel stenosis or valve stenosis, restenosis, deep vein thrombosis (DVT), pulmonary embolism, and ischemic or hemorrhagic stroke.

In embodiments of this aspect, the nucleic acid expression cassette, the vector, or the pharmaceutical composition is for use in the treatment of a lysosomal storage disease. In further embodiments, the disease or disorder is a lysosomal storage disease selected from the group comprising Fabry disease, glycogen storage disorders (e.g. Pompe disease, glycogen storage disorder (GSD) type II, Danon disease, GSD type IIb, GSD III or GSD 3 (also known as Cori's disease or Forbes' disease), GSD IV or GSD4 (also known as Andersen disease), GSD V or GSD5 (also known as McArdle disease), GSD VII or GSD7 (also known as Tarui's disease), GSD X or GSD10, GSD XII or GSD 12 (also known as Aldolase A deficiency), GSD XIII or GSD13, GSD XV or GSD15), and mucopolysaccharidosis disorders (e.g. Hunter syndrome, Sanfilippo syndrome, mucopolyssacharidose (MPS) I, MPS II, MPS III, MPS IIIA, MPS IIIB, MPS IIIC, MPS IV, MPS VI, MPS VII, MPS IX).

Aspect 24: the nucleic acid regulatory element according to any one of aspects 1 to 5, the nucleic acid expression cassette according to any one of aspects 7 to 15, the vector according to any one of aspects 16 to 18, or the pharmaceutical composition according to aspect 19 for use as a vaccine, preferably a prophylactic vaccine, or for use in vaccination therapy, preferably prophylactic vaccination.

Aspect 25: A method, preferably an in vitro or ex vivo method, for expressing a transgene product in heart and/or muscle cells or tissue, preferably in heart and muscle cells or tissue, more preferably in cardiomyocytes, comprising:
introducing the nucleic acid expression cassette according to any one of aspects 7 to 15, or the vector according to any one of aspects 16 to 18 into the heart and/or muscle cells or tissue, preferably into heart and muscle cells or tissue, more preferably into the cardiomyocytes;
expressing the transgene product in the heart and/or muscle cells or tissue, preferably in the heart and muscle cells or tissue, more preferably in the cardiomyocytes; and
optionally recovering the transgene product from the heart and/or muscle cells or tissue, preferably the cardiomyocytes, or the culture medium.

Aspect 26: An in vitro or ex vivo method for the production of antibodies or nanobodies in heart and/or muscle cells or tissue, preferably in cardiomyocytes, comprising:
introducing the nucleic acid expression cassette according to any one of aspects 7 to 15, or the vector according to any one of aspects 16 to 18, wherein the transgene encodes an antibody or a nanobody into the heart and/or muscle cells or tissue, preferably into the cardiomyocytes;
expressing the antibody or the nanobody in the heart and/or muscle cells or tissue, preferably in the cardiomyocytes; and
optionally recovering the antibody or the nanobody from the heart and/or muscle cells or tissue, preferably the cardiomyocytes, or the culture medium.

### BRIEF DESCRIPTION OF DRAWINGS

FIG. 1 shows a schematic representation of an AAV2 vector plasmid containing the cardiomyocyte-targeted SPc5-12 promoter driving the luciferase reporter (Luc2) gene. The identified CARD-CRE regulatory elements were cloned upstream of the SPc5-12 promoter. The AAV2 vector plasmid further comprises a polyadenylation site (pA) and minute virus of mouse (MVM) intron. The AAV vector plasmid is flanked by the 5′ and 3′ inverted terminal repeats (ITR).

FIG. 2 shows a schematic illustration of the in vivo screening and validation platform used for screening the identified CARD-CREs. CARD-CREs were cloned upstream of a cardiomyocyte-targeted promoter (e.g. SPc5-12 synthetic promoter) that drives expression of a luciferase reporter (Luc2) gene. Control vectors were devoid of any CARD-CRE. The corresponding vector constructs were packaged into AAV9 particles that were injected in CB17 SCID mice. Luciferase activity was quantified by in vivo bioluminescence imaging (BLI) or by luminometry on purified cardiomyocytes from the injected mice. Luciferase activity of the CARD-CRE vectors was compared side by side to compare robustness of the CARD-CREs to augment luciferase activity, or compared to a control vector without CARD-CRE or a CMV reference construct.

FIG. 3: Comparison of luciferase activity by whole body bioluminescence (BLI). Whole body BLI of CB17 SCID mice that were, 1 week earlier, injected with AAV9 viral vectors containing CARD-CRE3, CARD-CRE11, CARD-CRE12 or a control AAV9 viral vector devoid of CARD-CRE. The area indicated in white depicts luciferase activity.

FIG. 4: Comparison of luciferase activity by luminometry. Luminometric analysis in freshly purified cardiomyocytes of mice injected with an AAV9 viral vector containing CARD-CRE11 or a control AAV9 viral vector devoid of a CARD-CRE. An AAV viral vector encoding the luciferase reporter gene driven from a cytomegalovirus (CMV) promoter was used as a reference vector. Luciferase activity was expressed as relative luminescence Unit (RLU).

FIGS. 5A-5C: Comparison of luciferase activity by whole body BLI two weeks post vector injection (FIG. 5A) or by BLI analysis of individual organs and tissues harvested from dissected mice three weeks post vector injection (FIG. 5B) in CB17 SCID mice injected with AAV9 viral vectors containing CARD-CRE11 regulatory element, a control AAV9 viral vector which is devoid of a CARD-CRE or an AAV9 viral vector containing the CMV promoter as reference. The area indicated in white depicts luciferase activity. (FIG. 5C) Quantitative analysis of luciferase activity by BLI analysis in heart and muscle groups.

FIGS. 6A-6C: Comparison of luciferase activity by whole-body BLI one week post vector injection (FIG. 6A) or by BLI analysis of individual organs and tissues harvested from dissected mice 22 weeks post vector injection (FIG. 6B) in CB17 SCID mice injected with AAV9 viral vectors containing CARD-CRE14, CARD-CRE16 or CARD-CRE17 or a control AAV9 viral vector devoid of a CARD-CRE (no CRE control). The area indicated in white depicts luciferase activity. (FIG. 6C) Quantitative analysis of luciferase activity by BLI analysis in heart and muscle groups.

FIGS. 7A-7D: Comparison of luciferase activity by whole-body BLI 12 weeks post vector injection (FIG. 7A) or by BLI analysis of individual organs and tissues harvested from dissected mice 14 weeks post vector injection (FIG. 7B) in CB17 SCID mice injected with AAV9 viral vectors containing CARD-CRE8, CARD-CRE11, CARD-CRE16 or CARD-CRE20 or a control AAV9 viral vector devoid of a CARD-CRE (no CRE control). The area indicated in white depicts luciferase activity. (FIG. 7C) Quantitative analysis of luciferase activity by BLI analysis in heart and muscle groups. (FIG. 7D) Quantitative analysis of luciferase activity by BLI analysis in heart, different muscle groups and organs, 14 weeks post vector injection, in CB17 SCID mice injected with AAV9 viral vectors containing (a) CARD-CRE16, (b) CARD-CRE11, (c) CARD-CRE8 or (d) CARD-CRE20 or a control AAV9 viral vector which is devoid of CARD-CRE (no CRE control) (e).

FIG. 8: Vector map of the AAVss-hMLC-Luc2-SynpA vector.

FIG. 9: Vector map of the AAVss-Card CRE11-hMLC-Luc2-SynpA vector

FIG. 10: Luciferase activity in individual dissected tissues 4 weeks post vector injection. CB17-SCID mice were injected with ssAAV9-Card CRE11-hMLC-Luc2-SynpA (Card CRE11-hMLC) or ssAAV9-hMLC-Luc2-SynpA (hM LC) (vector dose: 10¹¹ vg per mouse). Luciferase expression was measured as total flux in photons/sec/cm²/sr (mean+s.e.m.; n=3).

FIG. 11: Vector map of the AAVss-CMV-Luc2-SynpA vector.

FIG. 12: Vector map of the AAVss-CARD CRE11-CMV-Luc2-SynpA vector.

FIG. 13: Luciferase activity in individual dissected heart 4 weeks post vector injection. CB17-SCID mice were injected with ssAAV9-Card CRE11-CMV-Luc2-SynpA (Card CRE11-CMV) or control vector devoid of CARD-CRE11 (ssAAV9-CMV-Luc2-SynpA; CMV) (vector dose: 10E11 vg per mouse). Luciferase expression was measured as total flux in photons/sec/cm2/sr (mean+s.e.m.; n=3).

### DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any or etc. of said members, and up to all said members.

In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

For general methods relating to the invention, reference is made inter alio to well-known textbooks, including, e.g., “Molecular Cloning: A Laboratory Manual, 4^{th} Ed.” (Green and Sambrook, 2012, Cold Spring Harbor Laboratory Press), “Current Protocols in Molecular Biology” (Ausubel et al., 1987).

In an aspect, the invention relates to a nucleic acid regulatory element for enhancing heart- and/or muscle-targeted gene expression, in particular heart- and muscle-targeted gene expression, more particularly cardiomyocyte-targeted gene expression, comprising, consisting essentially of (i.e., the regulatory element may for instance additionally comprise sequences used for cloning purposes, but the indicated sequences make up the essential part of the regulatory element, e.g. they do not form part of a larger regulatory region such as a promoter), or consisting of a sequence selected from the group consisting of: SEQ ID NO: 2, SEQ ID NO:3, SEQ ID NO:5, SEQ ID NO:6, SEQ ID NO: 7, SEQ ID NO: 8, a sequence having at least 80%, preferably at least 85%, more preferably at least 90%, even more preferably at least 95%, such as 95%, 96%, 97%, 98%, or 99%, identity to (the full-length of) any of these sequences, or a functional fragment thereof (i.e. a functional fragment of a sequence selected from the group consisting of: SEQ ID NO: 2, SEQ ID NO:3, SEQ ID NO:5, SEQ ID NO:6, SEQ ID NO: 7, SEQ ID NO: 8, or of a sequence having at least 80%, preferably at least 85%, more preferably at least 90%, even more preferably at least 95%, such as 95%, 96%, 97%, 98%, or 999% identity to (the full-length of) any of said sequences).

In certain embodiments, the nucleic acid regulatory elements described herein consist of a sequence selected from the group consisting of: SEQ ID NO: 2, SEQ ID NO:3, SEQ ID NO:5, SEQ ID NO:6, SEQ ID NO: 7 and SEQ ID NO: 8, and at least one flanking nucleotide sequence (i.e. a 5′ flanking nucleotide sequence and/or a 3′ flanking nucleotide sequence), wherein said at least one flanking nucleotide sequence has a maximal length of 50 nucleotides, preferably a maximal length of 45, 40, 35, 30 or 25 nucleotides, more preferably a maximal length of 20, 19, 18, 17, 16, 15, 14, 13, 12, 11, 10, 9, 8, 7, 6, 5, 4, 3, 2 or 1 nucleotides. In certain embodiments, the nucleic acid regulatory elements described herein consist of a sequence selected from the group consisting of: SEQ ID NO: 2, SEQ ID NO:3, SEQ ID NO:5, SEQ ID NO:6, SEQ ID NO: 7 and SEQ ID NO: 8, and two flanking nucleotide sequences located on opposite ends of said sequence selected from the group consisting of: SEQ ID NO: 2, SEQ ID NO:3, SEQ ID NO:5, SEQ ID NO:6, SEQ ID NO: 7 and SEQ ID NO: 8 (or a 5′ flanking nucleotide sequence and a 3′ flanking nucleotide sequence), wherein each flanking nucleotide sequence independently has a maximal length of 50 nucleotides, preferably a maximal length of 45, 40, 35, 30 or 25 nucleotides, more preferably a maximal length of 20, 19, 18, 17, 16, 15, 14, 13, 12, 11, 10, 9, 8, 7, 6, 5, 4, 3, 2 or 1 nucleotides. In embodiments, the flanking nucleotide sequence is a heterologous sequence (e.g. sequences used for cloning purposes). In other embodiments, the flanking nucleotide sequence is a homologous sequence.

Within the meaning of the present invention, “heterologous” in relation to a given sequence is understood as meaning any nucleic acid sequence other than those which are immediately adjacent to the said sequence in nature. The term “homologous” as used herein in relation to a given sequence is understood as meaning a nucleic acid sequence which is immediately adjacent to the said sequence in nature.

A ‘regulatory element’ or ‘nucleic acid regulatory element’, also referred to as “CRE” (cis-regulatory element), “CRM” (cis-regulatory module), or “CARD-CRE” or “CARD CRE” (cardiomyocyte-derived CRE), as used herein, refers to a transcriptional control element, in particular a non-coding cis-acting transcriptional control element, capable of regulating and/or controlling transcription of a gene, in particular tissue- or cell-targeted transcription of a gene. Regulatory elements comprise at least one transcription factor binding site (TFBS), in particular at least one binding site for a tissue- or cell-targeted transcription factor, more in particular at least one binding site for a heart- and/or muscle-targeted transcription factor, more particularly at least one binding site for a heart- and muscle-targeted transcription factor, most particularly at least one binding site for a cardiomyocyte-targeted transcription factor. Typically, regulatory elements as used herein increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g. in the promoter region) or downstream (e.g. in the 3′UTR) of the gene they regulate in vivo, and may be located in the immediate vicinity of the gene or further away or even within the gene itself. Of note, although regulatory elements as disclosed herein typically comprise naturally occurring sequences, combinations of (parts of) such regulatory elements or several copies of a regulatory element, i.e. regulatory elements comprising non-naturally occurring sequences, are themselves also envisaged as regulatory element. Regulatory elements as used herein may comprise part of a larger sequence involved in transcriptional control, e.g. part of a promoter sequence. However, regulatory elements alone are typically not sufficient to initiate transcription, but require a promoter to this end. The regulatory elements disclosed herein are provided as nucleic acid molecules, i.e. isolated nucleic acids, or isolated nucleic acid molecules. Said nucleic acid regulatory element hence have a sequence which is only a small part of the naturally occurring genomic sequence and hence is not naturally occurring as such, but is isolated therefrom.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alio pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA/RNA hybrid molecules, specifically including hnRNA, pre-mRNA, guide(g) RNA, mRNA, cDNA, non-coding (nc)-RNA, long non-coding RNA (Inc)RNA, short-hairpin (sh)RNA, small interfering (si)RNA, micro(mi)RNA, circular (c)RNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

Sequences disclosed herein may be part of sequences of regulatory elements capable of controlling transcription of genes in cardiomyocytes, in particular normal human cardiomyocytes, in vivo, more particularly controlling the following genes: tissue inhibitor of matrix metalloproteinases 1 (TIMP1 or TIMP-1) also known as CLGI, EPA, EPO or HCl; Collagen Type VI Alpha 2 Chain (COL6A2) also known as PP3610, BTHLM1 or UCMD1; Collagen Type I Alpha 2 Chain (COL1A2) also known as 014, EDSCV or EDSARTH2; galectin 1 (LGALS1) also known as GAL1 or GBP; Insulin Like Growth Factor Binding Protein 7 (IGFBP7) also known as AGM, FSTL2, IBP-7, IGFBP-7, IGFBP-7v, IGFBPRP1, MAC25, PSF, RAMSVPS or TAF; and fibronectin 1 (FN1) also known as CIG, ED-B, FINC, FN, FNZ, GFND, GFND2, LETS, MSF or SMDCF.

Accordingly, in embodiments, the nucleic acid regulatory elements disclosed herein comprise a sequence from TIMP1 regulatory elements, i.e. regulatory elements that control expression of the TIMP1 gene in vivo, e.g. regulatory elements comprising SEQ ID NO:2 (e.g. CARD-CRE8) or functional fragments thereof. In embodiments, the nucleic acid regulatory elements disclosed herein comprise a sequence from COL6A2 regulatory elements, i.e. regulatory elements that control expression of the COL6A2 gene in vivo, e.g. regulatory elements comprising SEQ ID NO:3 (e.g. CARD-CRE11), or functional fragments thereof. In embodiments, the nucleic acid regulatory elements disclosed herein comprise a sequence from COL1A2 regulatory elements, i.e. regulatory elements that control expression of the COL1A2 gene in vivo, e.g. regulatory elements comprising SEQ ID NO:5 (e.g. CARD-CRE14), or functional fragments thereof. In embodiments, the nucleic acid regulatory elements disclosed herein comprise a sequence from LGALS1 regulatory elements, i.e. regulatory elements that control expression of the LGALS1 gene in vivo, e.g. regulatory elements comprising SEQ ID NO:6 (e.g. CARD-CRE16) or SEQ ID NO:7 (e.g. CARD-CRE17), or functional fragments thereof. In embodiments, the nucleic acid regulatory elements disclosed herein comprise a sequence from IGFBP7 regulatory elements, i.e. regulatory elements that control expression of the IGFBP7 gene in vivo, e.g. regulatory elements comprising SEQ ID NO:8 (e.g. CARD-CRE20), or functional fragments thereof.

As used herein, the terms “identity” and “identical” and the like refer to the sequence similarity between two polymeric molecules, e.g., between two nucleic acid molecules, e.g., two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250). Typically, the percentage sequence identity is calculated over the entire length of the sequence. As used herein, the term “substantially identical” denotes at least 90%, preferably at least 95%, such as 95%, 96%, 97%, 98% or 99%, sequence identity.

The term ‘functional fragment’ as used in the application refers to fragments of the regulatory element sequences disclosed herein that retain the capability of regulating heart- and/or muscle-targeted expression, in particular heart- and muscle-targeted expression, more particularly of regulating cardiomyocyte-targeted expression, i.e. they can still confer targeted expression in a specific tissue or cell and they are capable of regulating expression of a (trans)gene in the same way (although possibly not to the same extent) as the sequence from which they are derived. Functional fragments as defined herein preferably have at least 50%, at least 60%, at least 70%, at least 80%, at least 90%, at least 95% or about 100% of the regulatory capacities of the nucleic acid regulatory element sequence from which they are derived. Functional fragments may preferably comprise at least 20, at least 25, at least 30, at least 35, at least 40, at least 45, at least 50, at least 60, at least 70, at least 80, at least 90, at least 100, at least 120, at least 150, at least 200, at least 250, at least 300, at least 350, or at least 400 contiguous nucleotides from the sequence from which they are derived. Also, functional fragments may comprise at least 1, more preferably at least 2, at least 3, or at least 4, even more preferably at least 5, at least 10, or at least 15, of the transcription factor binding sites (TFBS) that are present in the sequence from which they are derived.

As used herein “heart- and/or muscle-targeted expression” refers to the preferential or predominant expression of a (trans)gene (as RNA and/or polypeptide) in heart and/or muscle cells or heart and/or muscle tissue, in particular in heart muscle cells or tissue, skeletal muscle cells or tissue and/or diaphragm cells or tissue, more particularly in heart muscle cells, skeletal muscle cells and/or diaphragm cells, as compared to other (i.e. non-heart and/or -muscle) cells and tissues. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within heart and/or muscle cells or tissue. According to a particular embodiment, heart- and/or muscle-targeted expression entails that there is less than 10%, less than 5%, less than 2% or even less than 1% ‘leakage’ of expressed (trans)gene product to other organs, tissue or cells than heart and/or muscle cells and tissue, such as lung, liver, brain, kidney and/or spleen.

As used herein “heart-targeted expression” refers to the preferential or predominant expression of a (trans)gene (as RNA and/or polypeptide) in heart cells or tissue, in particular in heart muscle cells or tissue, more particularly in heart muscle cells, as compared to other (i.e. non-heart) cells and tissues. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within heart cells or tissue. According to a particular embodiment, heart-targeted expression entails that there is less than 10%, less than 5%, less than 2% or even less than 1% ‘leakage’ of expressed (trans)gene product to other organs, tissue or cells than heart, such as lung, liver, brain, kidney and/or spleen.

As used herein “muscle-targeted expression” refers to the preferential or predominant expression of a (trans)gene (as RNA and/or polypeptide) in muscle cells or tissue, in particular in skeletal muscle cells or tissue and/or diaphragm cells or tissue, more particularly in skeletal muscle cells and/or diaphragm cells, as compared to other (i.e. non-muscle) cells and tissues. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within muscle cells or tissue. According to a particular embodiment, heart-targeted expression entails that there is less than 10%, less than 5%, less than 2% or even less than 1% ‘leakage’ of expressed (trans)gene product to other organs, tissue or cells than muscle, such as lung, liver, brain, kidney and/or spleen.

As used herein “heart- and muscle-targeted expression” refers to the preferential or predominant expression of a (trans)gene (as RNA and/or polypeptide) in heart and muscle cells or tissue, in particular in heart muscle cells or tissue, skeletal muscle cells or tissue and diaphragm cells or tissue, more particularly in heart muscle cells, skeletal muscle cells and diaphragm cells, as compared to other (i.e. non-heart and -muscle) cells and tissues. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within heart and muscle cells or tissue. According to a particular embodiment, heart- and muscle-targeted expression entails that there is less than 10%, less than 5%, less than 2% or even less than 1% ‘leakage’ of expressed (trans)gene product to other organs, tissue or cells than heart and muscle, such as lung, liver, brain, kidney and/or spleen.

The same applies mutatis mutandis for (cardio)myocyte-targeted, (cardiac) muscle progenitor/stem cell-targeted, (cardiac) muscle satellite cell-targeted, and (cardiac) myoblast-targeted expression, which may be considered as a particular form of heart- and/or muscle-targeted expression. Throughout the application, where muscle-targeted is mentioned in the context of expression, (cardio)myocyte-targeted, (cardiac) muscle progenitor/stem cell-targeted, (cardiac) muscle satellite cell-targeted and (cardiac) myoblast-targeted expression are also explicitly envisaged. Similarly, where heart-targeted expression is used in the application, cardiomyocyte-targeted, cardiac muscle stem/progenitor cell-targeted, cardiac muscle satellite cell-targeted and cardiac myoblast-targeted expression is also explicitly envisaged.

The term “muscle” as used herein includes, without limitation, heart muscle tissue, skeletal muscle tissue and diaphragm, preferably skeletal muscle tissue and diaphragm.

As used herein, the terms “heart muscle” or “cardiac muscle” refer to the autonomically regulated, striated muscle tissue found in the heart.

As used herein, the term “skeletal muscle” refers to the voluntarily controlled, striated muscle tissue that is attached to the skeleton. Non-limiting examples of skeletal muscle tissue include the biceps, the triceps, the quadriceps, the tibialis, and the gastrocnemius muscle.

As used herein, the term “diaphragm” refers to a sheet of muscle tissue between the thorax and the abdomen.

The term “muscle cell” refers to any myocyte or muscle precursor cell (including myoblasts, muscle progenitor/stem cells and muscle satellite cells) for any type of muscle tissue (including smooth muscle, skeletal muscle, cardiac muscle and diaphragm), but excluding other non-muscle cells that are present in muscle tissue (e.g., endothelial cells, fibroblasts, pericytes and neurons). The term “myocyte,” as used herein, refers to a cell that has been differentiated from a progenitor myoblast such that it is capable of expressing muscle-specific phenotype under appropriate conditions. Terminally differentiated myocytes fuse with one another to form myotubes, a major constituent of muscle fibers. The term “myocyte” also refers to myocytes that are de-differentiated. The term includes cells in vivo and cells cultured ex vivo regardless of whether such cells are primary or passaged. The term “cardiomyocyte” as used herein particular refers to the myocytes that make up the heart muscle tissue.

The term “myoblast” as used herein, refers to an embryonic cell in the mesoderm that differentiates to give rise to a myocyte. The term includes cells in vivo and cells cultured ex vivo regardless of whether such cells are primary or passaged.

The term “muscle progenitor/stem cell” as used herein, refers to a cell that is capable to self-renew in addition to producing differentiated progeny within the myogenic lineage. A particular example of a muscle progenitor/stem cell is a muscle satellite cell. The term includes cells in vivo and cells cultured ex vivo regardless of whether such cells are primary or passaged.

In embodiments, the invention relates to a nucleic acid regulatory element for enhancing heart-and/or muscle-targeted gene expression, in particular heart- and muscle-targeted gene expression, more particularly cardiomyocyte-targeted gene expression, comprising, consisting essentially of, or consisting of a functional fragment of a sequence selected from the group consisting of: SEQ ID NO:2, SEQ ID NO:3, SEQ ID NO:5, SEQ ID NO:6, SEQ ID NO:7 or SEQ ID NO:8, a sequence having at least 80%, preferably at least 85%, more preferably at least 90%, even more preferably at least 95%, such as 95%, 96%, 97%, 98%, or 99%, identity to (the full-length of) any of these sequences, wherein said functional fragment comprises at least 20, preferably at least 25, more preferably at least 50, at least 100, at least 200 or at least 250, contiguous nucleotides from the sequence from which it is derived. In further embodiments, said functional fragment comprise at least 1, preferably at least 5, more preferably at least 10 or at least 15, of the transcription factor binding sites (TFBS) that are present in the sequence from which it is derived.

In further embodiments, the invention provides a nucleic acid regulatory element for enhancing heart- and/or muscle-targeted gene expression, in particular heart- and skeletal muscle-targeted gene expression, more particularly cardiomyocyte-targeted gene expression, comprising, consisting essentially of, or consisting of a sequence selected from the group consisting of: SEQ ID NO:2, SEQ ID NO:3, SEQ ID NO:5, SEQ ID NO:6, SEQ ID NO:7, SEQ ID NO:8, or a sequence having at least 80%, preferably at least 85%, more preferably at least 90%, even more preferably at least 95%, such as 95%, 96%, 97%, 98%, or 99%, identity to (the full-length of) any of these sequences.

It is also possible to make nucleic acid regulatory elements that comprise an artificial sequence by combining two or more identical or different nucleic acid regulatory element sequences disclosed herein or a functional fragment thereof. Accordingly, in certain embodiments a nucleic acid regulatory element for enhancing heart- and/or muscle-targeted gene expression, in particular heart- and muscle-targeted gene expression, more particularly cardiomyocyte-targeted gene expression, is provided comprising at least two sequences selected from the group consisting of: SEQ ID NO:2, SEQ ID NO:3, SEQ ID NO:5, SEQ ID NO:6, SEQ ID NO:7, SEQ ID NO:8, a sequence having at least 90%, preferably at least 95%, such as 95%, 96%, 97%, 98%, or 99%, identity to (the full-length of) any of these sequences, or a functional fragment thereof.

For example, disclosed herein is a nucleic acid regulatory element comprising, consisting essentially of, or consisting of 2, 3, 4, or 5 repeats, e.g. tandem repeats, of any one of SEQ ID NOs: 2, 3, 5, 6, 7 and 8, or combinations thereof. The repeats can be combined in tandem or with one or more such as 1, 2, 3, 4, 5, 6, 7, 8, 9, 10 or more intervening or flanking nucleotides (e.g. nucleotides used for cloning purposes) between one or more of the repeats.

In case the regulatory element is provided as a single stranded nucleic acid, e.g. when using a single-stranded AAV vector, the complement strand is considered equivalent to the disclosed sequences. Hence, also disclosed herein is a nucleic acid regulatory element for enhancing heart- and/or muscle-targeted gene expression, in particular heart- and muscle-targeted gene expression, more particularly cardiomyocyte-targeted gene expression, comprising, consisting essentially of, or consisting of the complement of a sequence described herein, in particular a sequence selected from the group consisting of: SEQ ID NO:2, SEQ ID NO:3, SEQ ID NO:5, SEQ ID NO:6, SEQ ID NO: 7, SEQ ID NO: 8, a sequence having at least 80%, preferably at least 85%, more preferably at least 90%, even more preferably at least 95%, such as 95%, 96%, 97%, 98%, or 99%, identity to (the full-length) any of these sequences, or a functional fragment thereof.

Also disclosed herein is a nucleic acid regulatory element for enhancing heart- and/or muscle-targeted gene expression, in particular heart- and muscle-targeted gene expression, more particularly cardiomyocyte-targeted gene expression, hybridizing under stringent conditions to a nucleic acid regulatory element described herein, in particular to the nucleic acid regulatory element comprising, consisting essentially of, or consisting of a sequence selected from the group consisting of: SEQ ID NO: 2, SEQ ID NO:3, SEQ ID NO:5, SEQ ID NO:6, SEQ ID NO: 7, SEQ ID NO: 8, a sequence having at least 90%, preferably at least 95%, such as 95%, 96%, 97%, 98%, or 99%, identity to (the full-length of) any of these sequences, a functional fragment thereof, or to its complement. Said nucleic acid regulatory elements do not need to be of equal length as the sequence they hybridize to. In preferred embodiments, the size of said hybridizing nucleic acid regulatory element does not differ more than 25% in length, in particular 20% in length, more in particular 15% in length, most in particular 10% in length from the sequence it hybridizes to.

The expression ‘hybridize under stringent conditions’, refers to the ability of a nucleic acid molecule to hybridize to a target nucleic acid molecule under defined conditions of temperature and salt concentration. Typically, stringent hybridization conditions are no more than 25° C. to 30° C. (for example, 20° C., 15° C., 10° C. or 5° C.) below the melting temperature (Tm) of the native duplex. Methods of calculating Tm are well known in the art. By way of non-limiting example, representative salt and temperature conditions for achieving stringent hybridization are: 1×SSC, 0.5% SDS at 65° C. The abbreviation SSC refers to a buffer used in nucleic acid hybridization solutions. One liter of the 20× (twenty times concentrate) stock SSC buffer solution (pH 7.0) contains 175.3 g sodium chloride and 88.2 g sodium citrate. A representative time period for achieving hybridization is 12 hours.

Preferably the nucleic acid regulatory elements as described herein are fully functional while being only of limited length. This allows their use in vectors or nucleic acid expression cassettes without unduly restricting their payload capacity. Accordingly, in embodiments, the nucleic acid regulatory element disclosed herein is a nucleic acid of 2000 nucleotides or less, 1800 nucleotides or less, 1700 nucleotides or less, 1500 nucleotides or less, 1000 nucleotides or less, 900 nucleotides or less, 800 nucleotides or less, 700 nucleotides or less, more preferably 600 nucleotides or less, such as 550 nucleotides or less, 500 nucleotides or less, 450 nucleotides or less, 400 nucleotides or less, 350 nucleotides or less, or 300 nucleotides or less (i.e. the nucleic acid regulatory element has a maximal length of 2000 nucleotides, 1800 nucleotides, 1700 nucleotides, 1500 nucleotides, 1000 nucleotides, 900 nucleotides, 800 nucleotides, 700 nucleotides, preferably 600 nucleotides, such as 550 nucleotides, 500 nucleotides, 450 nucleotides, 400 nucleotides, 350 nucleotides, or 300 nucleotides; or the nucleic acid regulatory element is maximal 2000 nucleotides, 1800 nucleotides, 1700 nucleotides, 1500 nucleotides, 1000 nucleotides, 900 nucleotides, 800 nucleotides, 700 nucleotides, preferably 600 nucleotides, such as 550 nucleotides, 500 nucleotides, 450 nucleotides, 400 nucleotides, 350 nucleotides, or 300 nucleotides long; or a nucleic acid regulatory element of 2000 nucleotides or less, 1800 nucleotides or less, 1700 nucleotides or less, 1500 nucleotides or less, 1000 nucleotides or less, 900 nucleotides or less, 800 nucleotides or less, 700 nucleotides or less, more preferably 600 nucleotides or less, such as 550 nucleotides or less, 500 nucleotides or less, 450 nucleotides or less, 400 nucleotides or less, 350 nucleotides or less, or 300 nucleotides or less).

However, it is to be understood that the disclosed nucleic acid regulatory elements retain regulatory activity (i.e. with regard to specificity and/or activity of transcription) and thus they particularly have a minimum length of 20 nucleotides, 25 nucleotides, 30 nucleotides, 35 nucleotides, 40 nucleotides, 45 nucleotides, 50 nucleotides, 100 nucleotides, 150 nucleotides, 200 nucleotides, 250 nucleotides, 300 nucleotides, 350 nucleotides or 400 nucleotides.

The nucleic acid regulatory elements disclosed herein may be used in a nucleic acid expression cassette. Accordingly, in an aspect the invention provides for the use of the nucleic acid regulatory elements as described herein in a nucleic acid expression cassette.

In an aspect the invention provides a nucleic acid expression cassette comprising a nucleic acid regulatory element as described herein, operably linked to a promoter. In embodiments, the nucleic acid expression cassette does not contain a transgene. Such nucleic acid expression cassette may be used to drive expression of an endogenous gene. In preferred embodiments, the nucleic acid expression cassette comprises a nucleic acid regulatory element as described herein, operably linked to a promoter and a transgene.

As used herein, the term ‘nucleic acid expression cassette’ refers to nucleic acid molecules that include one or more transcriptional control elements (such as, but not limited to promoters, enhancers and/or regulatory elements, polyadenylation sequences, and introns) that direct (trans)gene expression in one or more desired cell types, tissues or organs. Typically, they will also contain a transgene, although it is also envisaged that a nucleic acid expression cassette directs expression of an endogenous gene in a cell into which the nucleic acid cassette is inserted.

The term ‘operably linked’ as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and (a coding sequence of) a gene of interest to be expressed (i.e., the transgene), including transgenes expressing non-coding RNA as defined elsewhere herein. The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, the regulatory elements will typically be located immediately upstream of the promoter (although this is generally the case, it should definitely not be interpreted as a limitation or exclusion of positions within the nucleic acid expression cassette), but this needs not be the case in vivo. E.g., a regulatory element sequence naturally occurring downstream of a gene whose transcription it affects is able to function in the same way when located upstream of the promoter. Hence, according to a specific embodiment, the regulatory or enhancing effect of the regulatory element is position-independent.

In particular embodiments, the nucleic acid expression cassette comprises one nucleic acid regulatory element as described herein. In alternative embodiments, the nucleic acid expression cassette comprises two or more, such as, e.g., 2, 3, 4, 5, 6, 7, 8, 9, or 10, nucleic acid regulatory elements as described herein, i.e. they are combined modularly to enhance their regulatory (and/or enhancing) effect. In further embodiments, at least two of the two or more nucleic acid regulatory elements are identical or substantially identical. In yet further embodiments, all of the two or more regulatory elements are identical or substantially identical. The copies of the identical or substantially identical nucleic acid regulatory elements may be provided as tandem repeats in the nucleic acid expression cassette. In alternative further embodiments, at least two of the two or more nucleic acid regulatory elements are different from each other, that is to say, are defined by a different SEQ ID NO. The nucleic acid expression cassette may also comprise a combination of identical and substantially identical nucleic acid regulatory elements and non-identical nucleic acid regulatory elements. The at least two nucleic acid regulatory elements can be combined in tandem or they can be combined with one or more intervening or flanking nucleotides (e.g. nucleotides used for cloning purposes) between one or more of the regulatory elements.

As used in the application, the term ‘promoter’ refers to nucleic acid sequences that regulate, either directly or indirectly, the transcription of nucleic acid coding sequences to which they are operably linked (e.g. a transgene or endogenous gene). A promoter may function alone to regulate transcription or may act in concert with one or more other regulatory sequences (e.g. enhancers or silencers, or regulatory elements). In the context of the present application, a promoter is typically operably linked to a regulatory element as disclosed herein to regulate transcription of a (trans)gene. When a regulatory element as described herein is operably linked to both a promoter and a transgene, the regulatory element can (1) confer a significant degree of heart- and/or muscle-targeted, in particular heart- and muscle-targeted, more particularly cardiomyocyte-targeted, expression in vivo (and/or in heart and/or muscle cells or tissues, in particular in heart and muscle cells or tissue, more particularly in cardiomyocytes, or cell lines derived from heart and/or muscle cells or tissue in vitro) of the transgene, and/or (2) can increase the level of expression of the transgene in heart and/or muscle cells or tissue, in particular in heart and muscle cells or tissue, more particularly in cardiomyocytes (and/or in heart and/or muscle cells or tissue, in particular cardiomyocytes, or cell lines derived from heart and/or muscle cells or tissue in vitro).

The promoter may be homologous (i.e. from the same species as the animal, in particular mammal, to be transfected with the nucleic acid expression cassette) or heterologous (i.e. from a source other than the species of the animal, in particular mammal, to be transfected with the expression cassette). As such, the source of the promoter may be any virus, any unicellular prokaryotic or eukaryotic organism, any vertebrate or invertebrate organism, or any plant, or may even be a synthetic promoter (i.e. having a non-naturally occurring sequence), provided that the promoter is functional in combination with the regulatory elements described herein. In preferred embodiments, the promoter is a mammalian promoter, in particular a murine or human promoter.

Furthermore, the promoter does not need to be the promoter of the transgene in the nucleic acid expression cassette, although it is possible that the transgene is transcribed from its own promoter.

In embodiments, the promoter may be heterologous to the regulatory element, i.e. the promoter does not need to be the promoter of the regulatory element in the nucleic acid expression cassette.

The promoter may be an inducible or constitutive promoter.

The nucleic acid regulatory elements disclosed herein in principle direct heart- and/or muscle-targeted, in particular heart- and muscle-targeted, more particularly cardiomyocyte-targeted, expression even from a promoter that itself is not heart- and/or muscle-specific (e.g. CAG promoter, CMV promoter). Hence, the regulatory elements disclosed herein can be used in nucleic acid expression cassettes in conjunction with any promoter, in particular the promoter may either be tissue-specific, e.g. heart- and/or muscle-specific, or ubiquitously expressed.

Non-limiting examples of ubiquitously expressed promoters include the cytomegalovirus (CMV) promoter, RNA polymerase II (pol II) promoters, RNA polymerase III (pol III) promoters (e.g. an U6 pol III promoter, in particular the human U6 small nuclear promoter (U6), or a H1 pol III promoter, in particular the human H1 promoter (H1)) and chimeric pol III promoters. In particular embodiments, the promoter is the CMV promoter as defined by SEQ ID NO:15

(ACGCGTGGAGCTAGTTATTAATAGTAATCAATTACGGGGTCATTAGTT
CATAGCCCATATATGGAGTTCCGCGTTACATAACTTACGGTAAATGGCC
CGCCTGGCTGACCGCCCAACGACCCCCGCCCATTGACGTCAATAATGAC
GTATGTTCCCATAGTAACGTCAATAGGGACTTTCCATTGACGTCAATGG
GTGGAGTATTTACGGTAAACTGCCCACTTGGCAGTACATCAAGTGTATC
ATATGCCAAGTACGCCCCCTATTGACGTCAATGACGGTAAATGGCCCGC
CTGGCATTATGCCCAGTACATGACCTTATGGGACTTTCCTACTTGGCAG
TACATCTACGTATTAGTCATCGCTATTACCATGGTGATGCGGTTTTGGC
AGTACATCAATGGGCGTGGATAGCGGTTTGACTCACGGGGATTTCCAAG
TCTCCACCCCATTGACGTCAATGGGAGTTTGTTTTGCACCAAAATCAAC
GGGACTTTCCAAAATGTCGTAACAACTCCGCCCCATTGACGCAAATGGG
CGGTAGGCGTGTACGGTGGGAGGTCTATATAAGCAGAGCTCGTTTAGTG
AACCGTCAGATCGCCTGGAGACGCCATCCACGCTGTTTTGACCTCCATA
GAAGACACCGGGACCGATCCAGCCTCC)

These ubiquitously expressed promoters may be suitable for expressing non-coding RNA. In embodiments, the promoter is a RNA polymerase promoter, preferably a RNA polymerase II (pol II) promoter or a RNA polymerase III (pol III) promoter.

In embodiments, the nucleic acid expression cassettes disclosed herein comprise a heart- and/or muscle-targeted promoter, more preferably a heart- and muscle-targeted promoter. A heart-and/or muscle-targeted promoter as used herein refers to a promoter which preferentially or predominantly expresses a (trans)gene in heart and/or muscle cells or tissue and/or expression of the (trans)gene in other (i.e. non-heart and/or -muscle) cells and tissues is minimal. A heart- and muscle-targeted promoter as used herein refers to a promoter which preferentially or predominantly expresses a (trans)gene in heart and muscle cells or tissue and/or expression of the (trans)gene in other (i.e. non-heart and -muscle) cells and tissues is minimal. The use of a heart-and/or muscle-targeted promoter, in particular a heart- and muscle-targeted promoter, may increase targeted expression in heart- and/or muscle, in particular targeted expression in heart-and muscle, and/or avoid or reduce leakage of (trans)gene expression in other tissues or cells. Non-limiting examples of heart and/or muscle-targeted promoters include the desmin (DES) promoter; the alpha-actinl promoter (ACTA1); the Creatine kinase, muscle (CKM) promoter; the Four and a half LIM domains protein 1 (FHL1) promoter; the alpha 2 actinin (ACTN2) promoter; the filamin-C (FLNC) promoter; the sarcoplasmic/endoplasmic reticulum calcium ATPase 1 (ATP2A1) promoter; the Troponin I type 1 (TNNI1) promoter; the Troponin I type 2 (TNN12) promoter; the Troponin T type 1 (TNNT1) promoter; the Troponin T type 2 (TNNT2) promoter; the Troponin T type 3 (TNNT3) promoter; the myosin-1 (MYH1) promoter; the myosin-2 (MYH2) promoter; the sarcolipin (SLN) promoter; the Myosin Binding Protein C1 (MYBPC1) promoter; the enolase (EN03) promoter; the Carbonic Anhydrase 3 (CA3) promoter; the phosphorylatable, fast skeletal muscle myosin light chain (MYLPF) promoter; the Tropomyosin 1 (TPM1) promoter; the Tropomyosin 2 (TPM2) promoter; the alpha-3 chain tropomyosin (TPM3) promoter; the ankyrin repeat domain-containing protein 2 (ANKRD2) promoter; the myosin heavy-chain (MHC) promoter; the alpha myosin heavy chain promoter (ocMHC) promoter; the myosin light-chain (MLC) promoter such as the human myosin light-chain (hMLC) promoter; the muscle creatine kinase (MCK) promoter; the Myosin, Light Chain 1 (MYL1) promoter; the Myosin, Light Chain 2 (MYL2) promoter; the Myoglobin (MB) promoter; the Troponin C type 1 (TNNC1) promoter; the Troponin C Type 2 (TNNC2) promoter; the Titin-Cap (TCAP) promoter; the Myosin, Heavy Chain 7 (MYH7) promoter; the Aldolase A (ALDOA) promoter; the myosin heavy chain 11 (Myh11) promoter; the transgelin (Tagln) promoter (also known as SM22c promoter); the actin alpha 2, smooth muscle (Acta2) promoter; the SPc5-12 promoter; the dMCK promoter; the tMCK promoter; and the MHCK7 promoter.

In preferred embodiments, the promoter is a heart and/or muscle-targeted promoter selected from the group consisting of: the hMLC promoter, the SPc5-12 promoter, the DES promoter and the MHCK7 promoter. In embodiments, the promoter is a heart and/or muscle-targeted promoter selected from the group consisting of: the SPc5-12 promoter, the DES promoter and the MHCK7 promoter. The SPc5-12 promoter (sometimes called SPC-5-12-GTRM, which term is used interchangeabley herein) is a synthetic promoter and has been described in Li et al. (1999. Nat Biotechnol. 17:241-245). In particular embodiments, the promoter is the SPc5-12 promoter as defined by SEQ ID NO:11

### (TGGCCACCGCCTTCGGCACCATCCTCACGACACCCAAATATGGCGACGGGTGAGGAATGGTGGGGAGTTA

### TTTTTAGAGCGGTGAGGAAGGTGGGCAGGCAGCAGGTGTTGGCGCTCTAAAAATAACTCCCGGGAGTTAT

### TTTTAGAGCGGAGGAATGGTGGACACCCAAATATGGCGACGGTTCCTCACCCGTCGCCATATTTGGGTGTC

### CGCCCTCGGCCGGGGCCGCATTCCTGGGGGCCGGGCGGTGCTCCCGCCCGCCTCGATAAAAGGCTCCGGG

GCCGGCGGCGGCCCACGAGCTACCCGGAGGAGCGGGAGGCGCCAAGCTCTAGA). The MHCK7 promoter is a synthetic skeletal and cardiac muscle-targeted promoter and has been described in Salva et al. (2007. Mol Ther 15: 320-9). In particular embodiments, the promoter is the hMLC promoter as defined by SEQ ID NO:14

### (ATGAGCCACAGTGCCAGCCTTCATGGTTATTTTAAAGATGGTGGTCGGGGAGGCTTCACTCAGGAGATGA

### CATATGAGCAAAGATGCAGTGAAGGAGGTGAAGGAAGGAGCCGTGCGATGACTGACAGAAAGACATTCC

### AGGTAGAGGGCACACAGGTGCAAAGACCCTGAGGCCAGATCCAGGCTGATAAAACAGAGCATTTTAGCA

### GTCTCCTCTCCCTGCCATTTTTTTTCTCAAAATTGACAAGCACAAGTGTCCCCGGCCCAAGCACCGCAGAGA

### GCGCGCAGCATCTCTCCCCGTGACCATGACCCAGCTACTGCCTCTTTAACCTTGAATGCCTTTTTGGGGGCT

### CACGTGTCACCCAGTGGCGAGTGAGCCACCCTTACTTCAGAAGAACGGCATGGGGTGGGGGGGCCTTAGG

### TGGTGCCCGCCTCACCTATGACTGCCAAAAGCGGTCATGGGGTTATTTTTAAACATGGGGAGGAAGTATTT

### ATTGTTCCTGGGCTGCAGAGAGCTGGGCGGAGTGTGGAATTCTTCTCGGGAGGCAGTGCTGGGTCCTTTCC

### ACC)

In embodiments, the promoter is a mammalian heart- and/or muscle-targeted promoter, in particular a murine or human heart- and/or muscle-targeted promoter. In embodiments, the promoter is a synthetic heart- and/or muscle-targeted promoter. Non-limiting examples of synthetic heart- and/or muscle-targeted promoters have been described in Li et al. (1999, Nat Biotechnol. 17:241-245) and include, for example, the SPc5-12 promoter, the dMCK promoter, the tMCK promoter and the MHCK7 promoter. The dMCK and tMCK promoters consist of respectively, a double or triple tandem of the MCK enhancer to the MCK basal promoter as described in Wang et al. (2008, Gene Ther, 15:1489-1499).

To minimize the length of the nucleic acid expression cassette, the regulatory elements may be linked to minimal promoters, or shortened versions of the promoters described herein. A ‘minimal promoter’ (also referred to as basal promoter or core promoter) as used herein is part of a full-size promoter still capable of driving expression, but lacking at least part of the sequence that contributes to regulating (e.g. tissue-targeted) expression. This definition covers both promoters from which (tissue-targeted) regulatory elements have been deleted—that are capable of driving expression of a gene but have lost their ability to express that gene in a tissue-targeted fashion and promoters from which (tissue-targeted) regulatory elements have been deleted that are capable of driving (possibly decreased) expression of a gene but have not necessarily lost their ability to express that gene in a tissue-targeted fashion. Preferably, the promoter contained in the nucleic acid expression cassette disclosed herein is 1000 nucleotides or less in length, 900 nucleotides or less, 800 nucleotides or less, 700 nucleotides or less, 600 nucleotides or less, 500 nucleotides or less, 400 nucleotides or less, 300 nucleotides or less, or 250 nucleotides or less.

The term ‘transgene’ as used herein refers to particular nucleic acid sequences encoding a polypeptide or a portion of a polypeptide to be expressed in a cell into which the nucleic acid sequence is introduced. However, it is also possible that transgenes are expressed as non-coding RNA, e.g. to control (e.g. lower) the amount of a particular polypeptide in a cell into which the nucleic acid sequence is inserted.

How the nucleic acid sequence is introduced into a cell is not essential to the invention, it may for instance be through integration in the genome or as an episomal plasmid. Of note, expression of the transgene may be restricted to a subset of the cells into which the nucleic acid sequence is introduced. The term ‘transgene’ is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced.

The transgene may be homologous or heterologous to the promoter (and/or to the animal, in particular mammal, in which it is introduced, e.g. in cases where the nucleic acid expression cassette is used for gene therapy).

The transgene may be a full length cDNA or genomic DNA sequence, or any fragment, subunit or mutant thereof that has at least some biological activity. In particular, the transgene may be a minigene, i.e. a gene sequence lacking part, most or all of its intronic sequences. The transgene thus optionally may contain intron sequences. Optionally, the transgene may be a hybrid nucleic acid sequence, i.e., one constructed from homologous and/or heterologous cDNA and/or genomic DNA fragments. By ‘mutant form’ is meant a nucleic acid sequence that contains one or more nucleotides that are different from the wild-type or naturally occurring sequence, i.e., the mutant nucleic acid sequence contains one or more nucleotide substitutions, deletions, and/or insertions. The nucleotide substitution, deletion, and/or insertion can give rise to a gene product (i.e. e., protein or nucleic acid) that is different in its amino acid/nucleic acid sequence from the wild type amino acid/nucleic acid sequence. Preparation of such mutants is well known in the art. In some cases, the transgene may also include a sequence encoding a leader peptide or signal sequence such that the transgene product will be secreted from the cell.

In particular embodiments, the transgene is codon-optimized. As used herein the terms “codon-optimization”, “codon-optimized” and the like refer to changes in the codon composition of a nucleic acid sequence, in particular a transgene, without altering the amino acid sequence, e.g. for optimal expression in a host cell or organism. Codon-optimization of the transgene can further enhance heart- and muscle-targeted expression of the transgene.

The size of the transgenes contained in the nucleic acid expression cassettes and vectors described herein is not particularly limited, but may be determined by the packaging capacity of the vector as readily known to the skilled person. In embodiments, e.g. when using AAV vectors, the transgene contained in the nucleic acid expression cassette disclosed herein is less than 5 kb, such as less than 4 kb, less than 3 kb or less than 2 kb, in length. In embodiments, e.g. when using adenoviral vectors, the transgene contained in the nucleic acid expression cassette disclosed herein is less than 36 kb, such as less than 35 kb, less than 30 kb, less than 25 kb, less than 20 kb or less than 15 kb, in length. In embodiments, e.g. when using lentiviral vectors, the transgene contained in the nucleic acid expression cassette disclosed herein is less than 10 kb, such as less than 8 kb, less than 6 kb, less than 5 kb or less than 4 kb, in length.

The transgene that may be contained in the nucleic acid expression cassettes described herein typically encodes a gene product such as RNA or a polypeptide (protein).

In embodiments, the transgene encodes a non-coding RNA (ncRNA). As used herein, “non-coding RNA” refers to an RNA molecule that is transcribed from DNA but not translated into a protein. These non-coding RNA molecules include but are not limited to molecules that exert their function through RNA interference (e.g. short hairpin RNA (shRNA), small interfering RNA (siRNA)), micro-RNA regulation (miRNA) (which can be used to control expression of specific genes), long non-coding RNA (lncRNA), circular RNA (cRNA), catalytic RNA, antisense RNA, RNA aptamers, guide RNA used in the context of CRISPR systems, etc. In further embodiments, the transgene encodes a non-coding RNA selected from a microRNA, a long non-coding RNA, a circular RNA and a short interfering RNA.

In embodiments, the transgene encodes a therapeutic protein. Non-limiting examples of transgenes encoding a therapeutic protein include transgenes encoding angiogenic factors for therapeutic angiogenesis (e.g. VEGF, PIGF, or guidance molecules such as ephrins, semaphorins, Slits and netrins or their cognate receptors); transgenes encoding clotting factors (e.g. factor VIII or factor IX); transgenes encoding insulin; transgenes encoding lipoprotein lipase; transgenes encoding plasma factors; transgenes encoding cytokines, chemokines and/or growth factors (e.g. erythropoietin (EPO), interferon-α, interferon-β, interferon-γ, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C-X-C motif) ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1) and tumor necrosis factor (TNF)); transgenes encoding proteins involved in calcium handling (e.g. Sarco/Endoplasmic Reticulum Ca2+-ATPase (SERCA), phospholamban, calsequestrin, sodium-calcium exchanger, L-type calcium's channel, ryanodine receptors); transgenes encoding calcineurin; transgenes encoding microdystrophin; transgenes encoding follistatin (FST); transgenes encoding myotubularin 1 (MTM1); transgenes encoding dysferlin; transgenes encoding dystrophin; transgenes encoding metabolic enzymes; transgenes encoding nuclear proteins; transgenes encoding mitochondrial proteins (e.g. tafazzin); transgenes encoding lysosomal proteins (e.g. acid α-glucosidase (GAA) (as a secreted or native form), alpha-galactosidase A, LAM P2); transgenes encoding ion channels (e.g. SCNSA); transgenes encoding enzymes involved in glycogen metabolism (e.g. Glycogen synthase (GYS2), Glycogen debranching enzyme (AGL), Glycogen branching enzyme (GBE1), Muscle glycogen phosphorylase (PYGM), Muscle phosphofructokinase (PKFM), Phosphoglycerate mutase (PGAM2), Aldolase A (ALDOA), β-enolase (ENO3) or Glycogenin-1 (GYG1)); transgenes encoding enzymes deficient in mucopolysaccharidosis (e.g. α-L-iduronidase, Iduronate sulfatase, Heparan sulfamidase, N-acetylglucosaminidase, Heparan-α-glucosaminide N-acetyltransferase, N-acetylglucosamine 6-sulfatase, Galactose-6-sulfate sulfatase, β-galactosidase, N-acetylgalactosamine-4-sulfatase, β-glucuronidase or Hyaluronidase); transgenes encoding sarcoglycan (e.g. alpha-sarcoglycan, beta-sarcoglycan and gamma-sarcoglycan); transgenes encoding anoctamin 5; transgenes encoding calpain 3; transgenes encoding antibodies, transgenes encoding nanobodies, transgenes encoding anti-viral dominant-negative proteins; and transgenes encoding fragments, subunits or mutants of any of said therapeutic proteins. In particular embodiments, the transgene encodes acid α-glucosidase (GAA) (e.g. GAA as a secreted or native form). In particular embodiments, the transgene encodes a sarcoglycan, in particular a sarcoglycan selected from an alpha-sarcoglycan, a beta-sarcoglycan and a gamma-sarcoglycan, preferably a beta-sarcoglycan. In particular embodiments, the transgene encodes an antibody or a nanobody.

In embodiments, the transgene encodes an immunogenic protein. As used herein, the term “immunogenic” refers to a substance or composition capable of eliciting an immune response. Non-limiting examples of immunogenic proteins include epitopes and antigens derived from a pathogen.

Other sequences may be incorporated in the nucleic acid expression cassette disclosed herein as well, typically to further increase or stabilize the expression of the transgene product (e.g. introns and/or polyadenylation sequences).

Any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal. In embodiments, the nucleic acid expression cassette disclosed herein further comprises an intron. Non-limiting examples of suitable introns are a Minute Virus of Mice (MVM) intron, beta-globin intron (betalVS-II), factor IX (FIX) intron A, Simian virus 40 (SV40) small-t intron, and beta-actin intron.

Preferably, the intron is an MVM intron, more preferably the MVM intron defined by SEQ ID NO:12

### (AAGAGGTAAGGGTTTAAGGGATGGTTGGTTGGTGGGGTATTAATGTTTAATTACCTGGAGCACCTGCCTG

### AAATCACTTTTTTTTCAGGTTGG).

Any polyadenylation signal (also referred to herein as “polyadenylation site”, “poly A”, or “pA”) that directs the synthesis of a polyA tail is useful in the expression cassettes described herein, examples of those are well known to one of skill in the art. Exemplary polyadenylation signals include, but are not limited to, polyA sequences derived from the Simian virus 40 (SV40) late gene, the bovine growth hormone (BGH) polyadenylation signal, the minimal rabbit β-globin (mRBG) gene, and synthetic poly A sites (SPA or synt.pA), such as the synthetic pA site as described in Levitt et al. (1989, Genes Dev 3:1019-1025).

Preferably, the polyadenylation signal is a synthetic polyadenylation signal, more preferably the polyadenylation signal defined by SEQ ID NO:13

### (AATAAAAGATCTTTATTTTCATTAGATCTGTGTGTTG GIIIII1GTGTG).

In particular embodiments, the invention provides a nucleic acid expression cassette comprising a nucleic acid regulatory element comprising or consisting of a sequence selected from the group consisting of SEQ ID NO: 2, 3, 5, 6, 7 and 8, or a sequence having at least 95% identity to (the full length of) any one of said sequences, operably linked to a promoter, preferably the SPc5-12 promoter (preferably the SPc5-12 promoter as defined by SQ ID NO:11), and a transgene. In further embodiments, the nucleic acid expression cassette further comprises an MVM intron (preferably the MVM intron defined by SEQ ID NO:12). In yet further embodiments, the nucleic acid expression cassette further comprises a polyadenylation signal, preferably a synthetic polyadenylation signal, more preferably the polyadenylation signal defined by SEQ ID NO:13.

The nucleic acid regulatory element and the nucleic acid expression cassette disclosed herein may be used as such, or typically, they may be part of a nucleic acid vector. Accordingly, a further aspect relates to the use of a nucleic acid regulatory element as described herein or a nucleic acid expression cassette as described herein in a vector, in particular a nucleic acid vector.

In an aspect, the invention also provides a vector comprising a nucleic acid regulatory element as disclosed herein. In further embodiments, the vector comprises a nucleic acid expression cassette as disclosed herein.

The term ‘vector’ as used in the application refers to nucleic acid molecules, e.g. double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated. The vectors can be episomal vectors (i.e., that do not integrate into the genome of a host cell), or can be vectors that integrate into the host cell genome.

The term ‘vector’ may thus also be defined as a gene delivery vehicle that facilitates gene transfer into a target cell. This definition includes both non-viral and viral vectors. Non-viral vectors include but are not limited to cationic lipids, liposomes, nanoparticles, PEG, PEI, plasmid vectors (e.g. pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

In preferred embodiments, the vector is a viral vector, such as a retroviral, lentiviral, adenoviral, or adeno-associated viral (AAV) vector, preferably an AAV vector, a lentiviral vector or an adenoviral vector, more preferably an AAV vector. AAV vectors are preferably used as self-complementary, double-stranded AAV vectors (scAAV) in order to overcome one of the limiting steps in AAV transduction (i.e. single-stranded to double-stranded AAV conversion) (McCarty, 2001, 2003; Nathwani et al, 2002, 2006, 2011; Wu et al., 2008), although the use of single-stranded AAV vectors (ssAAV) are also encompassed herein.

Any AAV serotype can be used. The selection of an AAV serotype may be determined by the envisaged application and/or the host organism. In embodiments, the vector is an AAV serotype that achieves efficient transduction in heart and/or muscle tissue or cells, such as, e.g., AAV9. The vector may be an AAV vector of which the AAV capsid is engineered to direct the vector to a particular tissue or cell of interest such as to heart and/or muscle tissue or cells.

Production of AAV vector particles can be achieved e.g. by transient transfection of suspension-adapted mammalian HEK293 cells, as described (Chahal et al. Production of adeno-associated virus (AAV) serotypes by transient transfection of HEK293 cell suspension cultures for gene delivery, Journal of Virological Methods. 196: 163-173 (2014); Grieger et al., Production of recombinant adeno-associated virus vectors using suspension HEK293 cells and continuous harvest of vector from the culture media for GMP FIX and FLT1 clinical vector. Molecular Therapy. 24: 287-297 (2016); Blessing et al., Scalable Production of AAV Vectors in Orbitally Shaken HEK293 Cells. Molecular Therapy Methods & Clinical Development. 13: 14-26 (2019)), or by infection of Spodoptera frugiperda (Sf9) insect cells using the baculovirus expression vector system (BEVS), as described (Kotin et al. Manufacturing Clinical Grade Recombinant Adeno-Associated Virus Using Invertebrate Cell Lines. Human Gene Therapy. 28: 350-360 (2017)), followed by a purification step. Purification may be based on cesium chloride (CsCl) density gradient ultracentrifugation, as described (VandenDriessche et al., 2007), or using chromatographic techniques or columns or by immunoaffinity as known in the art.

In other embodiments, the vector is a non-viral vector, preferably a plasmid, a minicircle, or a transposon-based vector, such as a Sleeping Beauty (SB)-based vector or piggyBac (PB)-based vector.

In yet other embodiments, the vector comprises viral and non-viral elements.

In particular embodiments, a vector is provided comprising a nucleic acid expression cassette comprising a nucleic acid regulatory element comprising or consisting of a sequence selected from the group consisting of SEQ ID NO: 2, 3, 5, 6, 7 and 8, or a sequence having at least 95% identity to (the full-lenght of) any one of said sequences, a promoter, preferably the SPc5-12 promoter (more preferably the SPc5-12 promoter defined by SEQ ID NO:11), an MVM intron (preferably the MVM intron defined by SEQ ID NO:12), a transgene, and a polyadenylation signal, preferably a synthetic polyadenylation signal, more preferably the polyadenylation signal defined by SEQ ID NO:13.

The nucleic acid expression cassettes and vectors disclosed herein may be used, for example, to express proteins that are normally expressed and utilized in heart and/or muscle cell or tissue (i.e. structural proteins), or to express proteins that are expressed in muscle cell or tissue and that are then exported to the blood stream for transport to other portions of the body (i.e. secretable proteins). For example, the expression cassettes and vectors disclosed herein may be used to express a therapeutic amount of a gene product (such as a polypeptide, in particular a therapeutic protein, or RNA) for therapeutic purposes, in particular for gene therapy. Typically, the gene product is encoded by the transgene within the expression cassette or vector, although in principle it is also possible to increase expression of an endogenous gene for therapeutic purposes. In an alternative example, the expression cassettes and vectors disclosed herein may be used to express an immunological amount of a gene product (such as a polypeptide, in particular an immunogenic protein, or RNA) for vaccination purposes.

The nucleic acid expression cassettes and vectors as taught herein may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit.

The term “pharmaceutically acceptable” as used herein is consistent with the art and means compatible with the other ingredients of the pharmaceutical composition and not deleterious to the recipient thereof.

Accordingly, a further aspect of the invention relates to a pharmaceutical composition comprising a nucleic acid expression cassette or a vector described herein.

The use of nucleic acid regulatory elements described herein for the manufacture of these pharmaceutical compositions is also disclosed herein.

In embodiments, the pharmaceutical composition may be a vaccine. The vaccine may further comprise one or more adjuvants for enhancing the immune response. Suitable adjuvants include, for example, but without limitation, saponin, mineral gels such as aluminium hydroxide, surface active substances such as lysolecithin, pluronic polyols, polyanions, peptides, oil or hydrocarbon emulsions, bacilli Calmette-Guerin (BCG), Corynebacterium parvum, and the synthetic adjuvant QS-21. Optionally, the vaccine may further comprise one or more immunostimulatory molecules. Non-limiting examples of immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

In a further aspect, the invention relates to the nucleic acid regulatory elements, the nucleic acid expression cassettes, the vectors, or the pharmaceutical compositions described herein for use in medicine.

As used herein, the terms “treat” or “treatment” refer to both therapeutic treatment and prophylactic or preventative measures. Beneficial or desired clinical results include, but are not limited to, prevention of an undesired clinical state or disorder, reducing the incidence of a disorder, alleviation of symptoms associated with a disorder, diminishment of extent of a disorder, stabilized (i.e., not worsening) state of a disorder, delay or slowing of progression of a disorder, amelioration or palliation of the state of a disorder, remission (whether partial or total), whether detectable or undetectable, or combinations thereof. “Treatment” can also mean prolonging survival as compared to expected survival if not receiving treatment.

As used herein, the terms “therapeutic treatment” or “therapy” and the like, refer to treatments wherein the object is to bring a subjects body or an element thereof from an undesired physiological change or disorder to a desired state, such as a less severe or unpleasant state (e.g., amelioration or palliation), or back to its normal, healthy state (e.g., restoring the health, the physical integrity and the physical well-being of a subject), to keep it at said undesired physiological change or disorder (e.g., stabilization, or not worsening), or to prevent or slow down progression to a more severe or worse state compared to said undesired physiological change or disorder.

As used herein the terms “prevention”, “preventive treatment” or “prophylactic treatment” and the like encompass preventing the onset of a disease or disorder, including reducing the severity of a disease or disorder or symptoms associated therewith prior to affliction with said disease or disorder. Such prevention or reduction prior to affliction refers to administration of the nucleic acid regulatory elements, the nucleic acid expression cassettes, the vectors, or the pharmaceutical compositions described herein to a patient that is not at the time of administration afflicted with clear symptoms of the disease or disorder. “Preventing” also encompasses preventing the recurrence or relapse-prevention of a disease or disorder for instance after a period of improvement.

A further aspect relates to the nucleic acid regulatory elements, the nucleic acid expression cassettes, the vectors, or the pharmaceutical compositions described herein for use in gene therapy, in particular heart and/or muscle (cell or tissue)-directed gene therapy, more particularly heart and muscle (cell or tissue)-directed gene therapy.

Also disclosed herein is the use of the nucleic acid regulatory elements, the nucleic acid expression cassettes, the vectors, or the pharmaceutical compositions described herein for the manufacture of a medicament for gene therapy, in particular heart and/or muscle (cell or tissue)-directed gene therapy, more particularly heart and muscle (cell or tissue)-directed gene therapy.

Also disclosed herein is a method for gene therapy, in particular heart and/or muscle (cell or tissue)-directed gene therapy, more particularly heart and muscle (cell or tissue)-directed gene therapy, in a subject in need of said gene therapy comprising:
introducing in the subject, in particular in heart and/or muscle cells of the subject, more particularly in heart and muscle cells of the subjects, even more particularly in cadiomyocytes of the subject, a nucleic acid expression cassette, a vector or a pharmaceutical composition described herein, wherein the nucleic acid expression cassette, the vector or the pharmaceutical composition comprises a nucleic acid regulatory element described herein operably linked to a promoter and a transgene; and
expressing a therapeutically effective amount of the transgene product in the subject, in particular in heart and/or muscle cells of the subject, more particularly in heart and muscle cells of the subject, even more particularly in cardiomyocytes of the subject.

The transgene product may be a polypeptide, in particular a therapeutic protein. Non-limiting examples of therapeutic proteins have been disclosed above in connection with the transgene. The therapeutic protein may be a secretable protein. Non-limiting examples of secretable proteins, in particular secretable therapeutic proteins, include clotting factors, such as factor VIII or factor IX, secreted form of GAA, insulin, erythropoietin, lipoprotein lipase, antibodies or nanobodies, growth factors, cytokines, chemokines, enzymes, plasma factors, etc. The therapeutic protein may also be a non-secreted protein. Non-limiting examples of non-secreted proteins include intracellular proteins, metabolic enzymes (e.g. tafazzin), organel-targeted proteins (e.g. lysosomal proteins such as GAA, nuclear proteins), etc. The therapeutic protein may also be a structural protein. Non-limiting examples of structural proteins, in particular structural therapeutic proteins, include dystrophin and sarcoglycans.

Alternatively, the transgene product may be a non-coding RNA (ncRNA) molecule, such as an siRNA molecule, an miRNA molecule, a lncRNA molecule, a circRNA molecule, etc. as described above in connection with the transgene.

Exemplary diseases and disorders that may benefit from gene therapy, in particular heart and/or muscle (cell or tissue)—directed gene therapy using the nucleic acid regulatory elements, the nucleic acid expression cassettes, the vectors, or the pharmaceutical compositions described herein include:
cardiovascular diseases and heart diseases (e.g. atherosclerosis (which is defined as a disease in which plaque builds up inside the arteries causing a thickening and hardening of the artery walls); arteriosclerosis (which is defined as a disease in which plaque builds up on the inner walls of blood vessel lumen causing a narrowing of the lumen); coronary heart disease or coronary artery disease (which is defined as a disease for which the primary cause or pathogenesis is damage to the heart's major blood vessels caused primarily by atherosclerosis); peripheral arterial disease (which is defined as a disease for which the primary cause or pathogenesis is the narrowing of blood vessels caused by atherosclerosis that reduces blood flow to the limbs); congenital heart disease (which is defined as a disease for which the primary cause or pathogenesis is a structural abnormality in the heart that develops before birth); congestive heart failure, heart failure or cardiac insufficiency (which is defined as a disease that occurs when the heart is unable to pump enough blood flow to meet the body's needs for blood, oxygen and nutrients); myocardial infarction or heart attack (which is defined by the demonstration of myocardial cell necrosis typically due to significant and sustained ischemia); cardiac ischemia (which is defined as a disease that happens if blood supply to the myocardium does not meet the demand); acute coronary syndrome or unstable angina and stable angina (which is defined as a medical disorder characterized by ischemic chest pain or pressure usually due to an insufficient blood flow to the heart); cardiomyopathy (which is defined as a disease where the abnormal enlargement, or thickening, or rigidity of the heart muscle typically causes an insufficient coronary blood supply) such as hypertrophic cardiomyopathy, dilated cardiomyopathy (which is defined as a disease in which the heart cavities become enlarged and cannot pump blood effectively), restrictive cardiomyopathy (which is defined as a disease whereby the heart's ventricles become rigid because abnormal tissue, such as scar tissue, replaces the normal heart tissue) and, primary cardiomyopathies caused by genetic mutations, such as Brugada syndrome and Fabry disease; cardiac amyloidosis or “stiff heart syndrome” (which is defined as a disease that occurs when amyloid deposits take the place of normal heart muscle); myocarditis or inflammatory cardiomyopathy (which is defined as a disease characterized by inflammation and/or infection of the heart); valvular heart disease (which is defined as a disease involving one or more of the four valves of the heart (i.e. the aortic, mitral, pulmonary and tricuspid valves) which can be caused by valvular stenosis, valvular insufficiency, endocarditis or rheumatic heart disease (i.e. valvular damage caused by rheumatic fever)); pericarditis (which is defined as a disease caused by an inflammation and/or infection of the pericardium); cardiac tamponade, also known as pericardial tamponade (which is defined as a disease caused when fluid in the pericardium builds up, typically resulting in compression of the heart); endocarditis (which is defined as a disease caused by an inflammation and/or infection of the endocardium); cardiac arrhythmia (which is defined as a disease characterized by an abnormal heart rhythm) such as primary cardiac arrhythmias caused by genetic mutations, such as Brugada syndrome; hypertension (which is defined as a medical disorder characterized by abnormally high systolic or diastolic blood pressure); hypotension (which is defined as a medical disorder characterized by abnormally low systolic or diastolic blood pressure); vessel stenosis or valve stenosis (which is defined as a medical disorder characterized by an abnormal narrowing of a blood vessel or a heart valve orifice respectively); restenosis (which is defined as the recurrence of abnormal narrowing of an artery or valve after corrective surgery, including for example angioplasty); deep vein thrombosis (DVT) (which is defined as a disease characterized by the formation of a blood clot or thrombus in a vein deep in the body, typically in the legs); pulmonary embolism (which is defined as a blockage in one of the pulmonary arteries in the lungs typically secondary to DVT); and ischemic or hemorrhagic stroke (which is defined as a medical disorder in which poor blood flow to the brain results in cell death either due to lack of blood flow or bleeding));
lysosomal storage diseases (e.g. Fabry disease) including glycogen storage disorders (e.g. Pompe disease, glycogen storage disorder (GSD) type II, Danon disease, glycogen storage disorder (GSD) type Ilb, GSD III or GSD 3 (also known as Cori's disease or Forbes' disease), GSD IV or GSD4 (also known as Andersen disease), GSD V or GSD5 (also known as McArdle disease), GSD VII or GSD7 (also known as Tarui's disease), GSD X or GSD10, GSD XII or GSD 12 (also known as Aldolase A deficiency), GSD XIII or GSD13, GSD XV or GSD15) and mucopolysaccharidosis disorders (e.g. Hunter syndrome, Sanfilippo syndrome, mucopolyssacharidose (MPS) I, MPS II, MPS III, MPS IIIA, MPS IIIB, MPS IIIC,
MPS IV, MPS VI, MPS VII, MPS IX);
mitochondrial disorders (e.g. Barth syndrome);
channelopathy (e.g. Brugada syndrome);
metabolic disorders;
myotubular myopathy (MTM);
muscular dystrophy (e.g. Duchenne muscular dystrophy (DMD), Becker muscular dystrophy (BMD));
myotonic dystrophy; Myotonic Muscular Dystrophy (DM); Miyoshi myopathy;
Fukuyama type congenital;
dysferlinopathies;
neuromuscular disease;
motor neuron diseases (MND) (e.g. Charcot-Marie-Tooth disease (CMT), spinal muscular atrophy (SMA) or amyotrophic lateral sclerosis (ALS));
Emery-Dreifuss muscular dystrophy;
facioscapulohumeral muscular dystrophy (FSHD);
congenital muscular dystrophies;
congenital myopathies;
limb girdle muscular dystrophy (e.g. Limb Girdle Muscular Dystrophy type 2E (LGMD2E), Limb Girdle Muscular Dystrophy type 2D (LGMD2D), Limb Girdle Muscular Dystrophy type 2C (LGMD2C), Limb Girdle Muscular Dystrophy type 2B (LGMD2B), Limb Girdle Muscular Dystrophy type 2L (LGMD2L), Limb Girdle Muscular Dystrophy type 2A (LGMD2A));
metabolic myopathies;
muscle inflammatory diseases;
myasthenia;
mitochondrial myopathies;
anomalies of ionic channels;
nuclear envelop diseases;
distal myopathies;
coagulation disorders (e.g. hemophilia (e.g. hemophilia A and B), FVII deficiency, von Willebrand's disease);
diabetes;
C1 inhibitor deficiency or hereditary angioedema;
α1-antitrypsin deficiency;
kidney failure.

Diseases and disorders as described herein may have a cardiac and/or muscle dysfunction and benefit from heart and/or muscle (cell or tissue)-directed gene therapy as described herein. Depending on the dysfunction underlying the disease or the disorder, these diseases and disorders may particularly benefit from heart (cell or tissue)-directed gene therapy, in particular cardiomyocyte-directed gene therapy, or muscle (cell or tissue)-directed gene therapy, or both, as will be appreciated by the skilled person. These diseases and disorders may benefit from the expression of an appropriate therapeutic protein from the heart and/or muscle (cell or tissue), or from an appropriate non-coding RNA (e.g. siRNA, microRNA, lncRNA or circRNA).

Non-limiting examples of diseases or disorder that would particularly benefit from heart (cell or tissue)-directed gene therapy, in particular cardiomyocyte-directed gene therapy, include cardiovascular diseases and disorders such as atherosclerosis; arteriosclerosis; coronary heart disease or coronary artery disease; peripheral arterial disease; congenital heart disease; congestive heart failure, heart failure or cardiac insufficiency; myocardial infarction or heart attack; cardiac ischemia; acute coronary syndrome or unstable angina and stable angina; cardiomyopathy such as hypertrophic cardiomyopathy, dilated cardiomyopathy, restrictive cardiomyopathy and, primary cardiomyopathies caused by genetic mutations, such as Brugada syndrome and Fabry disease; cardiac amyloidosis or “stiff heart syndrome”; myocarditis or inflammatory cardiomyopathy; valvular heart disease; pericarditis; cardiac tamponade, also known as pericardial; endocarditis; cardiac arrhythmia such as primary cardiac arrhythmias caused by genetic mutations, such as Brugada syndrome; hypertension; hypotension; vessel stenosis or valve stenosis; and restenosis.

Non-limiting examples of diseases or disorder that would particularly benefit from muscle (cell or tissue)-directed gene therapy include lysosomal storage diseases (e.g. Fabry disease) including glycogen storage disorders (e.g. Pompe disease glycogen storage disorder (GSD) type II, Danon disease, GSD type Ilb, GSD III or GSD 3 (also known as Cori's disease or Forbes' disease), GSD IV or GSD4 (also known as Andersen disease), GSD V or GSD5 (also known as McArdle disease), GSD VII or GSD7 (also known as Tarui's disease), GSD X or GSD10, GSD XII or GSD 12 (also known as Aldolase A deficiency), GSD XIII or GSD13, GSD XV or GSD15) and mucopolysaccharidosis disorders (e.g. Hunter syndrome, Sanfilippo syndrome, mucopolyssacharidose (MPS) I, MPS II, MPS III, MPS IIIA, MPS IIIB, MPS IIIC, MPS IV, MPS VI, MPS VII, MPS IX); mitochondrial disorders (e.g. Barth syndrome); channelopathy (e.g. Brugada syndrome); metabolic disorders; myotubular myopathy (MTM); muscular dystrophy (e.g. Duchenne muscular dystrophy (DMD), Becker muscular dystrophy (BMD)); myotonic dystrophy; Myotonic Muscular Dystrophy (DM); Miyoshi myopathy; Fukuyama type congenital; dysferlinopathies; neuromuscular disease; motor neuron diseases (MND) (e.g. Charcot-Marie-Tooth disease (CMT)), spinal muscular atrophy (SMA) or amyotrophic lateral sclerosis (ALS)); Emery-Dreifuss muscular dystrophy; facioscapulohumeral muscular dystrophy (FSHD); congenital muscular dystrophies; congenital myopathies; limb girdle muscular dystrophy (e.g. Limb Girdle Muscular Dystrophy type 2E (LGMD2E), Limb Girdle Muscular Dystrophy type 2D (LGMD2D), Limb Girdle Muscular Dystrophy type 2C (LGMD2C), Limb Girdle Muscular Dystrophy type 2B (LGMD2B), Limb Girdle Muscular Dystrophy type 2L (LGMD2L), Limb Girdle Muscular Dystrophy type 2A (LGMD2A)); metabolic myopathies; muscle inflammatory diseases; myasthenia; mitochondrial myopathies; anomalies of ionic channels; nuclear envelop diseases; distal myopathies; cardiomyopathies; cardiac hypertrophy; heart failure; deep vein thrombosis (DVT); pulmonary embolism; and ischemic or hemorrhagic stroke.

Other diseases and disorder as described herein, such as, without limitation, coagulation disorders (e.g. hemophilia A and B, FVII deficiency, von Willebrand's disease), C1 inhibitor deficiency or hereditary angioedema, diabetes, al-antitrypsin deficiency and kidney failure, may benefit from heart and/or muscle (cell or tissue)-directed gene therapy as described herein, in particular muscle (cell or tissue)-directed gene therapy as disclosed herein through expression of a secretable therapeutic protein as described herein from muscle (cell or tissue), in particular skeletal muscle (cell or tissue).

A particular form of gene therapy disclosed herein encompasses antibody gene therapy. More particularly, provided herein are the nucleic acid expression cassettes, the vectors, or the pharmaceutical compositions comprising said nucleic acid expression cassettes or said vectors as described herein for use in a method of producing antibodies or nanobodies in a subject, preferably a human subject, the method comprising introducing the nucleic acid expression cassette or the vector into heart and/or muscle cells of the subject, preferably into heart and muscle cells of the subject, more preferably into cardiomyocytes of the subject in an amount effective to elicit expression of an antibody or a nanobody, wherein the transgene in the nucleic acid expression cassette or in the vector encodes the antibody or the nanobody.

In embodiments of this aspect, the subject is administered the nucleic acid expression cassette, the vector or the pharmaceutical composition. Methods for delivering nucleic acid expression cassettes and vectors to heart and/or muscle cell or tissue of the subject has been described elsewhere herein.

Introduction of the nucleic acid expression cassette or the vector into the heart and/or muscle cells of the subject can also be carried out in vitro. In embodiments, desired target heart and/or muscle cellss are removed from the subject, transfected or transduced with the nucleic acid expression cassette or the vector and reintroduced into the subject. Alternatively, syngeneic or xenogeneic heart and/or muscle cells can be used where those cells will not generate an inappropriate immune response in the subject.

Suitable methods for the transfection or transduction and reintroduction of transfected or transduced cells into a subject are known in the art. For example, heart and/or muscle cells can be transfected or transduced in vitro by combining the nucleic acid expression cassette or the vector with the heart and/or muscle cells, e.g., in appropriate media, and screening for those cells harboring the DNA of interest using conventional techniques such as Southern blots and/or PCR, or by using selectable markers. Transfected or transduced cells can then be formulated into a pharmaceutical composition, and the composition introduced into the subject by various techniques, such as by intramuscular, intravenous, subcutaneous and intraperitoneal injection, or by injection into smooth and/or cardiac muscle, using e g., a catheter. Accordingly, in embodiments, the method of producing antibodies or nanobodies in a subject further comprises a step of administering to the subject the heart and/or muscle cells, preferably the heart and muscle cells, more preferably the cardiomyocytes into which the nucleic acid expression cassette or the vector has been introduced.

A further aspect relates to the nucleic acid regulatory elements, the nucleic acid expression cassettes, the vectors, or the pharmaceutical compositions described herein for use in the treatment of a disease or disorder selected from the group comprising: cardiovascular diseases and disorders (such as atherosclerosis; arteriosclerosis; coronary heart disease or coronary artery disease; peripheral arterial disease; congenital heart disease; congestive heart failure, heart failure or cardiac insufficiency; myocardial infarction or heart attack; cardiac ischemia; acute coronary syndrome or unstable angina and stable angina; cardiomyopathy such as hypertrophic cardiomyopathy, dilated cardiomyopathy, restrictive cardiomyopathy and, primary cardiomyopathies caused by genetic mutations, such as Brugada syndrome and Fabry disease; cardiac amyloidosis or “stiff heart syndrome”; myocarditis or inflammatory cardiomyopathy; valvular heart disease; pericarditis; cardiac tamponade, also known as pericardial; endocarditis; cardiac arrhythmia such as primary cardiac arrhythmias caused by genetic mutations, such as Brugada syndrome; hypertension; hypotension; vessel stenosis or valve stenosis; restenosis; deep vein thrombosis (DVT); pulmonary embolism; and ischemic or hemorrhagic stroke); lysosomal storage diseases ((e.g. Fabry disease) including glycogen storage disorders (e.g. Pompe disease glycogen storage disorder (GSD) type II, Danon disease, GSD type IIb, GSD III or GSD 3 (also known as Cori's disease or Forbes' disease), GSD IV or GSD4 (also known as Andersen disease), GSD V or GSD5 (also known as McArdle disease), GSD VII or GSD7 (also known as Tarui's disease), GSD X or GSD10, GSD XII or GSD 12 (also known as Aldolase A deficiency), GSD XIII or GSD13, GSD XV or GSD15) and mucopolysaccharidosis disorders (e.g. Hunter syndrome, Sanfilippo syndrome, mucopolyssacharidose (MPS) I, MPS II, MPS III, MPS IIIA, MPS IIIB, MPS IIIC, MPS IV, MPS VI, MPS VII, MPS IX)); mitochondrial disorders (e.g. Barth syndrome); channelopathy (e.g. Brugada syndrome); metabolic disorders; myotubular myopathy (MTM); muscular dystrophy (e.g. Duchenne muscular dystrophy (DMD), Becker muscular dystrophy (BMD)); myotonic dystrophy; Myotonic Muscular Dystrophy (DM); Miyoshi myopathy; Fukuyama type congenital; dysferlinopathies; neuromuscular disease; motor neuron diseases (MND) (e.g. Charcot-Marie-Tooth disease (CMT)), spinal muscular atrophy (SMA) or amyotrophic lateral sclerosis (ALS)); Emery-Dreifuss muscular dystrophy; facioscapulohumeral muscular dystrophy (FSHD); congenital muscular dystrophies; congenital myopathies; limb girdle muscular dystrophy (e.g. Limb Girdle Muscular Dystrophy type 2E (LGMD2E), Limb Girdle Muscular Dystrophy type 2D (LGMD2D), Limb Girdle Muscular Dystrophy type 2C (LGMD2C), Limb Girdle Muscular Dystrophy type 2B (LGMD2B), Limb Girdle Muscular Dystrophy type 2L (LGMD2L), Limb Girdle Muscular Dystrophy type 2A (LGMD2A)); metabolic myopathies; muscle inflammatory diseases; myasthenia; mitochondrial myopathies; anomalies of ionic channels; nuclear envelop diseases; distal myopathies; coagulation disorders (e.g. hemophilia A and B, FVII deficiency, von Willebrand's disease), C1 inhibitor deficiency or hereditary angioedema, diabetes, al-antitrypsin deficiency and kidney failure.

Gene therapy protocols have been extensively described in the art. These include, but are not limited to, intramuscular injection of plasmid (naked or in liposomes), hydrodynamic gene delivery in various tissues, including muscle, interstitial injection, instillation in airways, application to endothelium, and intravenous or intra-arterial administration. Various devices have been developed for enhancing the availability of DNA to the target cell. A simple approach is to contact the target cell physically with catheters or implantable materials containing DNA. Another approach is to utilize needle-free, jet injection devices which project a column of liquid directly into the target tissue under high pressure. These delivery paradigms can also be used to deliver vectors. Another approach to targeted gene delivery is the use of molecular conjugates, which consist of protein or synthetic ligands to which a nucleic acid-or DNA-binding agent has been attached for the specific targeting of nucleic acids to cells (Cristiano et al., 1993).

A further aspect relates to the nucleic acid regulatory elements, the nucleic acid expression cassettes, the vectors, or the pharmaceutical compositions described herein for use as a vaccine, more particularly for use as a prophylactic vaccine.

Also disclosed herein is the use of the nucleic acid regulatory elements, the nucleic acid expression cassettes, the vectors, or the pharmaceutical compositions described herein for the manufacture of a vaccine, in particular for the manufacture of a prophylactic vaccine.

Also disclosed herein is a method of vaccination, in particular prophylactic vaccination, of a subject in need of said vaccination comprising:
introducing in the subject, in particular in heart and/or muscle cells of the subject, more particularly in cardiomyocytes, a nucleic acid expression cassette, a vector or a pharmaceutical composition described herein, wherein the nucleic acid expression cassette, the vector or the pharmaceutical composition comprises a nucleic acid regulatory element described herein operably linked to a promoter and a transgene; and
expressing an immunologically effective amount of the transgene product in the subject, in particular in heart and muscle of the subject.

As used herein, a phrase such as “a subject in need of treatment” includes subjects that would benefit from treatment of a recited disease or disorder. Such subjects may include, without limitation, those that have been diagnosed with said disease or disorder, those prone to contract or develop said disease or disorder and/or those in whom said disease or disorder is to be prevented.

The terms “subject” and “patient” are used interchangeably herein and refer to animals, preferably vertebrates, more preferably mammals, and specifically include human patients and non-human mammals. “Mammalian” subjects include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients or subjects are human subjects.

A ‘therapeutic amount’ or ‘therapeutically effective amount’ as used herein refers to the amount of gene product effective to treat a disease or disorder in a subject, i.e., to obtain a desired local or systemic effect. The term thus refers to the quantity of gene product that elicits the biological or medicinal response in a tissue, system, animal, or human that is being sought by a researcher, veterinarian, medical doctor or other clinician. Such amount will typically depend on the gene product and the severity of the disease, but can be decided by the skilled person, possibly through routine experimentation.

An “immunologically effective amount” as used herein refers to the amount of (trans)gene product effective to enhance the immune response of a subject against a subsequent exposure to the immunogen encoded by the (trans)gene. Levels of induced immunity can be determined, e.g. by measuring amounts of neutralizing secretory and/or serum antibodies, e.g., by plaque neutralization, complement fixation, enzyme-linked immunosorbent, or microneutralization assay.

Typically, the amount of (trans)gene product expressed when using an expression cassette or vector as described herein (i.e., with at least one heart- and muscle-targeted nucleic acid regulatory element) are higher than when an identical expression cassette or vector is used but without a nucleic acid regulatory element therein. The expression may be at least double as high, at least five times as high, at least ten times as high, at least 20 times as high, at least 30 times as high, at least 40 times as high, at least 50 times as high, or even at least 60 times as high as when compared to the same nucleic acid expression cassette or vector without nucleic acid regulatory element. Moreover, the higher expression preferably remains targeted to heart and muscle. Furthermore, the expression cassettes and vectors described herein can direct the expression of a therapeutic amount of the gene product for an extended period. Typically, therapeutic expression is envisaged to last at least 20 days, at least 50 days, at least 100 days, at least 200 days, and in some instances 300 days or more. Expression of the gene product (e.g. polypeptide) can be measured by any art-recognized means, such as by antibody-based assays, e.g. a Western Blot or an ELISA assay, for instance to evaluate whether therapeutic expression of the gene product is achieved. Expression of the gene product may also be measured in a bioassay that detects an enzymatic or biological activity of the gene product.

Also disclosed herein is the use of the nucleic acid regulatory elements, the nucleic acid expression cassettes, or the vectors disclosed herein for transfecting or transducing heart and muscle cells, preferably cardiomyocytes.

Further disclosed herein is the use of the nucleic acid expression cassettes or the vectors disclosed herein for expressing a transgene product in heart and/or muscle cells, preferably in cardiomyoctyes, wherein the nucleic acid expression cassette or the vector comprises a nucleic acid regulatory element disclosed herein operably linked to a promoter and a transgene.

Further disclosed herein is a method for expressing a transgene product in heart and/or muscle cells, preferably in cardiomyoctyes, comprising:
transfecting or transducing the heart and/or muscle cells, in particular the cardiomyocytes, with a nucleic acid expression cassette or a vector disclosed herein, wherein the nucleic acid expression cassette or the vector comprises a nucleic acid regulatory element disclosed herein operably linked to a promoter and a transgene; and
expressing the transgene product in the heart and/or muscle cells.

Expression of the transgene product in the heart and/or muscle cells may comprise culturing the heart and/or muscle cells or tissue, such as the cardiomyocytes, under suitable conditions to allow expression of the transgene product in the the heart and/or muscle cells or tissue, such as in the cardiomyocytes.

In further embodiments, the method may further comprise a step of recovering the transgene product from the heart and/or muscle cells or tissue, such as the cardiomyocytes, and/or the culture medium.

Non-viral transfection or viral vector-mediated transduction of heart and/or muscle cells, in particular cardiomyocytes, may be performed by in vitro, ex vivo or in vivo procedures. The in vitro approach requires the in vitro transfection or transduction of heart and/or muscle cells, e.g. heart and muscle cells previously harvested from a subject, heart and/or muscle cell lines or heart and/or muscle cells differentiated from e.g. induced pluripotent stem cells or embryonic cells. The ex vivo approach requires harvesting of the heart and/or muscle cells from a subject, in vitro transfection or transduction, and optionally re-introduction of the transfected heart and/or muscle cells into the subject. The in vivo approach requires the administration of the nucleic acid expression cassette or the vector disclosed herein into a subject. In preferred embodiments, the transfection of the heart and/or muscle cells is performed in vitro or ex vivo.

It is understood by the skilled person that the use of the nucleic acid regulatory elements, the nucleic acid expression cassettes and vectors disclosed herein has implications beyond gene therapy, e.g. coaxed differentiation of stem cells into cardiogenic or myogenic cells, transgenic models for over-expression of proteins in heart and muscle, etc.

The invention is further explained by the following non-limiting examples

### EXAMPLES

### Example 1: Identification of Normal Human Cardiomyocyte-Derived Nucleic Acid Regulatory Elements (CARD-CREs)

Materials and methods

Three samples of normal human cardiomyocyte-derived cell pellets were purchased from Promocell (Cat. #C-14080).

Total RNA was extracted from these cell pellets using AllPrep DNA/RNA Mini Kit (Qiagen, Germany) according to the manufacturer's instructions. Briefly,
1. 200 μl phosphate-buffered saline (PBS) was added into tubes containing 200 μl cell pellet (around 1×10⁶ cells).
2. The tubes were centrifuged at 500 g/4° C. in 10 min, the supernatant was removed.
3. 350 μl of RLT Plus buffer supplemented with 1% 2-mercaptoethanol was added, and pipetted several times to lyse the cells completely.
4. 350 μl of 70% ethanol was added and mixed well by pipetting. All solution was transferred to RNeasy spin column of AllPrep DNA/RNA Mini Kit and the manufacturer's instruction were followed as below:
5. The RNeasy spin column was centrifuged for 1 min at 13000 rpm and the flow-through was discarded.
6. 350 μl of RW1 buffer was added to RNeasy spin column. 7. The RNeasy spin column was centrifuged for 1 min at 13000 rpm and the flow-through was discarded.
8. 80 μl of DNase I in RDD buffer (Qiagen, Germany) was added directly to the RNeasy spin column and incubated at room temperature for 15 min.
9. 350 μl of RW1 buffer was added to RNeasy spin column.
10. The RNeasy spin column was centrifuged for 1 min at 13000 rpm and the flow-through was discarded.
11. 500 μl of RPE buffer was added to RNeasy spin column.
12. The RNeasy spin column was centrifuged for 1 min at 13000 rpm and the flow-through was discarded.

Expression analysis in normal human cardiomyocytes was determined by RNA sequencing (RNA-seq) (BGI tech solutions Ltd. (Hong Kong)). FPKM (i.e. fragments per kilobase of exon model per million reads mapped) reads corresponded to normalised gene expression levels. Six highly expressed genes based on RNA-seq analysis derived from normal human cardiomyocytes were selected for CRE identification: TIMP1, COL6A2, COL1A2, LGALS1, IGFBP7, FN1.

The transcriptional start sites of the selected genes were mapped using the UCSC Genome Brower Database. The nucleic acid elements were selected based on: 1) the presence of DNAse hypersensitivity sites; ii) high content of epigenetic markers associated with open chromatin; iii) high content of transcriptional factor binding sites (TFBS).

Results

Based on the above criteria, we selected nucleic acid regulatory elements, designated as Cis-Regulatory Elements (CREs) associated with high expression in cardiomyocytes (also designated herein as CARD-CRE) (Table 1). The location, sequence and the length of each of the CRE is also provided (Table 1).

TABLE 1
Cardiomyocyte-derived nucleic acid regulatory elements. Bp: base pairs.

| SEQ ID | | | Size | >location based on the hg19 genome browser database |
|---|---|---|---|---|
| NO | Gene | Name | (bp) | Sequence |
| SEQ ID | FN1 | CARD- | 482 | >hg19_dna range = chr2:216306183-216306664 strand = + |
| NO: 1 | | CRE3 | | CATTTTATTAAACACTCTAAACCAGATCCAGTACTAAGTGTTTCATA |
| | | | | TGAATTTTTTTCATCTAACCATCAACAAACTITTAAGGTAAGCACTA |
| | | | | TCATCCTCTCCTCTTACAGATGAGGATATAAAGCTTAGAAAAACAA |
| | | | | TGTGCAAAAATGCTACTCATTAAGCACAGATGTGAAAAGGAAAAA |
| | | | | GAAAATGCCCTGAAGGCAGCTCGCATGAGTATAATGTCTGAGAAG |
| | | | | TTTGTTGATAATTGACAAAGAATGAAGTTTTCTCAATCACAAGCCTT |
| | | | | GTAGAAATTGTGGCGTCAAGTTGTGTCACTGTCAATACCAGGTGAT |
| | | | | TAGTCAAGTGATTACATAATGGTCTCCCTCAGAACCAAAGGTATGG |
| | | | | GCAAGACAACTGGCTAAAGAATTTTGTGGGTGACTTCTTTCACATG |
| | | | | AGATTAATTTATCAAAAGATGTCACACAGGACTCTGCCAACTGACA |
| | | | | CGCTACCAGTTTCCATCTGTT |
| SEQ ID | TIMP1 | CARD- | 355 | >hg19_dna range = chrX:47441334-47441688 strand = + |
| NO: 2 | | CRE8 | | CTTATCTCCCTCCACTGCTGCTACTTAATTTGTTTCCTCTCTGCCACC |
| | | | | CCTCACCAGCATGTCAGACATACAAAACAAGGGATTTTTGTGTGCT |
| | | | | TGGCACACAGTAGATGCACAATAAATGTTGAAGGGCTGAACTAAT |
| | | | | TTGGGTTTGAGTCATAGGGAGCTTGGGGGATGTGGGTGATTGGAT |
| | | | | AGATTCTGGAGACTTTAGGGGACTGGGCCGGGGGAAATGCGGCCT |
| | | | | CTAAGCTCTCCGCTGAGGCGGCTTGGAAGGAATAGTGACTGACGT |
| | | | | GGAGGTGGGGGAGGTGGCTGGCCCGGGCGAGGCCCAGGGAGAG |
| | | | | GGAGAGGAGGCGGGTGGGAGAGGAGGAGGGTGTATCTC |
| SEQ ID | COL6A2 | CARD- | 1640 | >hg19_dna range = chr21:47511761-47513400 5′pad = 0 |
| NO: 3 | | CRE11 | | 3′pad = 0 strand = + repeatMasking = none |
| | | | | AACTGAGTTGTGTGACATTCACGTGGCCCCTTCAGTTAGCACCGCA |
| | | | | CGGTTGGTTAAGAGGAGGGTTTTATTCAGGCATAAATATTTTGAAA |
| | | | | TATTTGTAATCAAATCCCACGTAGTATAGACAACCTCCCAGATGGT |
| | | | | GGAGGGGAAACACACCACCCCCCTGACTGGGAGAGAAAAGACCG |
| | | | | CGGTTTTCTTTCCTGCCACTTGTCCTCCTCCGGGAGCCTCGGTCTCC |
| | | | | TCTCTGTGGGACAGGGCAGCCTCCCCTGGCTCCTCCTGGTGCAGGA |
| | | | | TCGATGAGGCTCTTGCCCACAGCCTTTGTCCCCCGACTTCCTACCAC |
| | | | | CTGGTGTCCTCACCCCATCTGGGCCCCGAGCACACAGCCTCTGGTC |
| | | | | CTGGTTTCCACTGAAAATGTAAACTATGGTCCACGCCACATTCTCCA |
| | | | | GTACTCCTGTGCTATTGTGAAAGTAAAAGACAAAAATACACGTGCT |
| | | | | TGGCAGCCCGAGAGCCTGGGTGAGATTATTGAGAATAATGCGGTG |
| | | | | GCTCTGGCTTAAAAAATAAAAGCCGGAGGATCTACCACCAGCTC |
| | | | | TGAACACACACCCGTGACACAAACTGACTCATGTGCAATTGCCCAT |
| | | | | TAGTGGCAGGTCTTGCCATTTTTCGTTTTTTTCTGAGTCGAGATCAG |
| | | | | CTGCGGCTGGGGAGAACATTAGTTCTGTCTGTCGGCTCCATGAGTA |
| | | | | AGCAGAGGAAAGTCTGACTACAGAGGCATCTTCTCAAATGTCCATG |
| | | | | AAAGGCTGCAAGCCCGAGAGTAACGCCCCTGCTGCCCTGAGTGGT |
| | | | | CCACGTGGTGTCCAGAGTGTGAGAAAGCCACAGAGAGCTTTTCCA |
| | | | | CACGGAGCCACTTAAGGCCTGCCTCAGCCTGAGAACACTTGGGCA |
| | | | | GAAAAGGACAGCACGGCTTCCTCCTCCTCCAGGCCGGGCCTACAA |
| | | | | GCACCCACAGTTCCGACAGAGAAAGTCTCTTCTTAATGGTACGCAT |
| | | | | GTGTCTTACTGTAGCTCCCAGTTAGTCCCCTAAGCGTGAACCACCAC |
| | | | | CCACCCATGCTGCTCCCCTCCCACCATCTCTCCATAAACAGGACCCA |
| | | | | ACGCAGCCAAAAACTCATGGTCATGTGACCTTGGCATCAGCTTGCT |
| | | | | CGTTAGGAACCGTGCCTGGAGGTGCTCTGAGCACTGACTGCCAAC |
| | | | | CTCAGCAGGGCCCTTTGTGGTGTTGAGGTGTGGTGTCCGCAGGGC |
| | | | | AGCCTGCAGCCTGCTGGGGTCCAGCCTGGGGCCTCAGGTCCTGGG |
| | | | | AACCCTGGTTGAGGCTGGGCTGCCTCAGTGCCCCTCCGCAGCCTGC |
| | | | | TGCACCCAAGCACTCTGAGAAAAGGAGCAATCACCCAACTTCAGTC |
| | | | | TGAAAATGAGAATGTTGAGAAGGGCATGATGAGGGGAAGCAGAC |
| | | | | CAGAGCCCACCGGCAAGGGTAGAACGTCAGCCCCGCCCTAGTGGG |
| | | | | ACAGACAAGAGAGAAGCCTCCTCACACCAGGAGGCAGTATAGCCG |
| | | | | GAGCCTTCCCTCCAGGGCTCTCAGGAGAGCCTGTGCTGGTTCATGA |
| | | | | AAAAGTGAGGCCTCTGGGCTTTGCTGCTGCAGAGTGAGGTCAGAG |
| | | | | CCCACAGGCCCCACAGATCATCCTGCCTGACAAGCACAGGAGGCA |
| | | | | CCACTGCTTGAATTCAGAGGGCAGCTCCGAGGGTCCTAGGTC |
| SEQ ID | COL6A2 | CARD- | 495 | >hg19_dna range = chr21:47511774-47512268 strand = + |
| NO: 4 | | CRE12 | | GACATTCACGTGGCCCCTTCAGTTAGCACCGCACGGTTGGTTAAGA |
| | | | | GGAGGGTTTTATTCAGGCATAAATATTTTGAAATATTTGTAATCAA |
| | | | | ATCCCACGTAGTATAGACAACCTCCCAGATGGTGGAGGGGAAACA |
| | | | | CACCACCCCCCTGACTGGGAGAGAAAAGACCGCGGTTTTCTTTCCT |
| | | | | GCCACTTGTCCTCCTCCGGGAGCCTCGGTCTCCTCTCTGTGGGACA |
| | | | | GGGCAGCCTCCCCTGGCTCCTCCTGGTGCAGGATCGATGAGGCTCT |
| | | | | TGCCCACAGCCTTTGTCCCCCGACTTCCTACCACCTGGTGTCCTCAC |
| | | | | CCCATCTGGGCCCCGAGCACACAGCCTCTGGTCCTGGTTTCCACTG |
| | | | | AAAATGTAAACTATGGTCCACGCCACATTCTCCAGTACTCCTGTGCT |
| | | | | ATTGTGAAAGTAAAAGACAAAAATACACGTGCTTGGCAGCCCGAG |
| | | | | AGCCTGGGTGAGATTATTGAGAATAATGCGGTGGC |
| SEQ ID | COL1A2 | CARD- | 548 | >hg19_dna range = chr7:94021125-94021672 strand = + |
| NO: 5 | | CRE14 | | CCCCACCCCACCCCTTACTTGTTTTTCCTTCTACTCATTTTCTCCTCCC |
| | | | | TCCCTCTCCCCTCTCCCCACACTAGAACCATCAGGGCCCTCACCAGG |
| | | | | GCATTCAGTTCAGGTTCTGGGTCAGCTCTCACAGCTCTACCAGTCCC |
| | | | | ACCCCCATCCCCAGGAAAAACTGCTCCTTATTTGGAGTCACAAAAA |
| | | | | TATTTAACAGAGATCTAACTGACCACTAAAAATTCCTCCTTTAAAAC |
| | | | | AAACACCTAATCAA |
| | | | | CTATTTTCCCCCAAGTTATATGGAAAAACAGCTGCAATTAGAACTTG |
| | | | | ATTCTCACTTTAAGAAAGAAAGATTCTTGTTTGGTTTTCTCCACTTTC |
| | | | | ATTTTTTGTTTCTAGGTCCAGGGCCTCCCACCAAATGCTGACGGCTG |
| | | | | CCTGCTTCAAACCCTGCCACATCAGCAGGGAGGGAGCACAGCGGC |
| | | | | ATTTGGTATTTGCATCAGTTTCCAGGAATGCTTTTCAAGTTATCAGT |
| | | | | TCACTCTGCTGCCTTTAGCAGAGACGTTTTCCCTCTAAGTTTATAGA |
| | | | | TGTCTGCTTCAATTTAC |
| SEQ ID | LGALS1 | CARD- | 844 | >hg19_dna range = chr22:38070761-38071604 strand = + |
| NO: 6 | | CRE16 | | AGCTGAGATCACGCCACTGCACTCCAGCCTGGGCGACAGAGCGAG |
| | | | | ACTCCATCTCAAAAATAAATATATAAATAAATAAAAATAAAAAAGA |
| | | | | GAAAGGACAGGGTGCACAGAGCCAGGACCCCACCCCCTCCTGGTC |
| | | | | TGGACCACCCTGGGCCATCTCCTAAGCTTGACACTCTCAGGCCTGA |
| | | | | GGCTGTCAGGCCAGGGCGCTTTGACTATCCCCTGCCTGCCCCTGAA |
| | | | | CGCGGCCAAGCCCGAGGCCTTAGCCAAGCCCTGCAGCCTCAGGCT |
| | | | | TGGCGCCCGGCCCAGCCTTTCTTTAGCCTTCCCGAGGAGCCAGCGA |
| | | | | GAGAGCGCTGCAGCAGCCGCAGTCCTTGCTTCCGGGGAAGGCCCG |
| | | | | GGTGGGGCCAGGGCCTGTGGTTAAGTCAGTGGCCCTCTGCAGACG |
| | | | | CGCACGCAGCAGGAGCCACGCCCTGGTCCCTGCCCGGGAGTGGCC |
| | | | | TGGTCTCCCCCAGCTCTGATTCCCTGAAGGACCCTGCACCTCCTCAG |
| | | | | CCATCTTCTCTGGGCACCCCCTGAGCCCAGCCTGCTGGCCACAGGC |
| | | | | CCCTTCCCGCCAACTTGTGGGCCTAGCTCATCATCAAACTATTTTCC |
| | | | | ACTGGCTTCCCCCAATCTCTGCCACAGCACAATTTTGGTGGGGAGG |
| | | | | TGGAGAGGGATGAAGGAGAATAGGAGGTGGAGCTTGGACTCTGG |
| | | | | CCGGGGGCGGCGGGGTGAGGGGGGGCAGCAGCTCGCCACTCTGA |
| | | | | TTGGTCACCTCTGCTCCAAAACTGGCTTCAAAATTCCACGGACTCCG |
| | | | | CCCCCAGTGGCCCCCAGCCCTATCCTGACTTGCAATTGGCTGAACTT |
| | | | | TCAGGGGGCGGGGCTCACCCGG |
| SEQ ID | LGALS1 | CARD- | 300 | >hg19_dna range = chr22:38071293-38071592 strand = + |
| NO: 7 | | CRE17 | | CTGCTGGCCACAGGCCCCTTCCCGCCAACTTGTGGGCCTAGCTCAT |
| | | | | CATCAAACTATTTTCCACTGGCTTCCCCCAATCTCTGCCACAGCACA |
| | | | | ATTTTGGTGGGGAGGTGGAGAGGGATGAAGGAGAATAGGAGGTG |
| | | | | GAGCTTGGACTCTGGCCGGGGGCGGCGGGGTGAGGGGGGGCAG |
| | | | | CAGCTCGCCACTCTGATTGGTCACCTCTGCTCCAAAACTGGCTTCAA |
| | | | | AATTCCACGGACTCCGCCCCCAGTGGCCCCCAGCCCTATCCTGACTT |
| | | | | GCAATTGGCTGAACTTTCAGGGGGCG |
| SEQ ID | IGFBP7 | CARD- | 1478 | >hg19_dna range = chr4:57976540-57978017 strand = + |
| NO: 8 | | CRE20 | | AAGGGCGCGAGTGAGCCGTGTCGGGCCGGCCGGCGCCTTAAACCC |
| | | | | GCCGCCCCGCCCGGCCTGTCCGCGGGAACCACACCCCGGGGCGGT |
| | | | | GAGAGCACGGGGGCCCCGCCCGTGCCGCCCTCCCGGCCCCGCGGG |
| | | | | GCCTGCGCGCGCGCTCCTGCTGGCGTCGGGGGGTGAGCGGGCACC |
| | | | | CGGCGTGACCGGCCCATGGGCGCTCGGTCTCACGCCCGCTTCGCA |
| | | | | GAGCTCACCCCGCCAGCTCCTGCTCGGTCCCCAGTCCCCTGCCCCG |
| | | | | AGCCCTCAGGGATCGCACCGCAGTGCGAGCGGCTCTTCCACCCTCT |
| | | | | GGTTTCTCCCCGTTTCCGGTCAATATATTTAAACTTCTTAACAACCAC |
| | | | | GGCTTCGAGACGATCCAGGGTTTCATCTATTAAATCCTCCGTGGGC |
| | | | | CTGAGACACCTGAGTGCTCATTACTCTGTGGTTTGGGAAGCTCTCTT |
| | | | | AAGAACAAGCTGTTAAAAACCCCCACTTGTGGACTGTGTCCCCGCT |
| | | | | GGCCACAGGCCTGCTGTGGTCTTGGGTGTCAAGCACTAAAAGGAC |
| | | | | AAACCGTTGCCAACCACTTCCCTTTCTGGAACCTCTTAACTGCCTGC |
| | | | | ACCTCCCTCCCCGCTTTGTCTCCCAGTGGCATTCAACAAGACTCTGT |
| | | | | GCTTTTTTTGTGCTGAGCCGTTTTCAGTGAGTACTGGAATCCGTGTG |
| | | | | AGAGCTGTGCCCTTGTTTTGAGTTCTTGCTGAACTGCTCGCCCTCCA |
| | | | | GCAAGCCTCTCGGGAGAACAGAGCGCAGCTGAGAGGAGCAGGGG |
| | | | | TCCCAGCTCCTTCCTCGTTGGAGCTGCCACGGAGGCACCTAAAGCG |
| | | | | CCCCCAGTCTTCACCAGCATTCCAGGAGGTTTTTGATATAAACGGT |
| | | | | GGACTCTTCCTCAGTCTTTCTGCGAAGTGCTCTGCAAACTCAACTTG |
| | | | | AAAGCCTCTCTTCCCAGGAACACTGGCCTCTCCAATGCAGACATGG |
| | | | | AAGTTGCTTTCACTTTTTAACTGAATGTGTAACATTGCAGCGTGCAT |
| | | | | TATTCTGCATGTCACATTGTGGTTCTTTTGTGTTCTTTTTTCTTAGCA |
| | | | | TAAATCCAGGGATTTACAGGGTTGCTGACTGAAAGCCAGCAGTTCA |
| | | | | GTTCATAATCCCATTAGCAGTGTAAAGAACTCATTCTCAACAGACTC |
| | | | | TTACAAGGATAATTATTGCAAATTTTTTGAAAATTAAATGACCACAA |
| | | | | AATCGGGGGGTGGAGAGTGGGGAAACATCCTCCAATCCAGAAAA |
| | | | | ACCTAGCACTGCTTTTTGGCCTCTCCTTAGTGTATTATAGACAAGTG |
| | | | | GAGCATTTTTTTTCCCCTCAAAGGTCATATGTTGACCTGACAGTTGT |
| | | | | TTAAAAAACAA |
| | | | | TGAAAAATCTTTACAGCAGAGTTTCCCTAAATATTTTTGTCTTTGTTC |
| | | | | CATCCTAAGAGATGCAGTTTACATGACCCATTTGTTTTCTTGAAAAG |
| | | | | GAAACAAATGCAGAGTGCACTCTATTTCCTATT |

### Example 2: In Vivo Validation of Identified Cardiomyocyte-Derived Nucleic Acid Regulatory Elements (CARD-CREs)

Materials and Methods

Cloning of Cardiomyocyte-Derived Cis-Regulatory Elements (CARD-CREs) into an AAV Vector

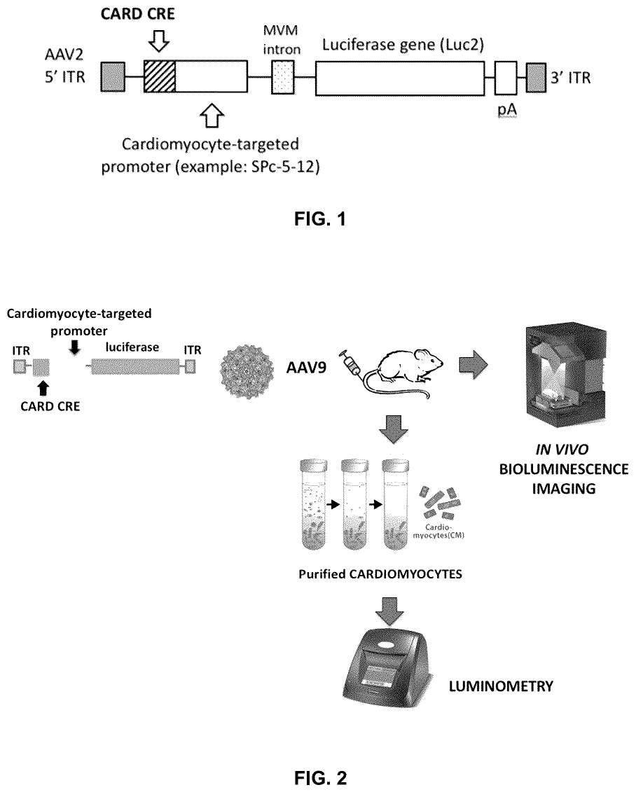

The CARD-CREs indicated in Table 1 were individually screened for their ability to enhance gene expression in an artificial synthetic context that is distinct from its natural genomic context. To do this, the CARD-CREs were first synthesized by conventional oligonucleotide synthesis, flanked with a MluI restriction site at the 5′ end and a BsiWI restriction site at the 3′ end for convenient cloning. These synthesized CRE fragments were then restricted with MluI and BsiWI and cloned into the AAV2 vector plasmid backbone restricted with Ascl at the 5′ end and Acc65I at the 3′ end. A sticky end cloning was possible since MluI/Asci and Acc65I/BsiWI generate compatible ends. The different CARD-CREs (SEQ ID NO: 1-8) were cloned upstream of the artificial heart and muscle directed promoter designated as SPc5-12 or Spc5-12GTRM (Li et al. 1999. Nat Biotechnol. 17(3):241-245) in a single stranded adeno-associated viral vector (AAV) backbone (FIG. 1). This promoter drove a reporter gene luciferase (Luc2). A synthetic polyadenylation site (poly A) (also designated herein as synt.pA) was cloned downstream of the Luc2 gene to ensure adequate transcriptional termination.

AAV Production

The AAV vectors were produced by calcium phosphate (Invitrogen Corp, Carlsbad, California, USA) co-transfection into AAV-293 cells (Stratagene, San Diego, California, USA) of the vector plasmid of interest, a chimeric packaging construct expressing AAV2 Rep and AAV9 Cap and an adenoviral helper plasmid, as previously described (Chuah et al. 2014. Mol Ther. 22(9):1605-1613; VandenDriessche et al. 2007. J Thromb Haemost. 5(1): 16-24). The capsid was derived from the AAV9 serotype given its cardiotropic and muscle-tropic properties (VandenDriessche et al. 2007, Sarcar et al. 2019. Nat Commun. 10(1):492). Two days after transfection, the cells were harvested and lysed by means of a combination of freeze/thaw cycles and sonication, a subsequent treatment with benzonase (Novagen, Madison, Wisconsin, USA) and deoxycholic acid (Sigma-Aldrich, St Louis, Missouri, USA), and 3 successive rounds of density gradient ultracentrifugation with cesium chloride (Invitrogen Corp, Carlsbad, California, USA). The AAV vector-containing fractions were collected and dialyzed in Dulbecco's phosphate buffered saline (PBS) (Gibco, BRL) containing 1 mM MgCl 2. The concentration of viral particles containing viral genomes, also called vector titers, were determined by quantitative real-time polymerase chain reaction (PCR) with SYBR® Green using luciferase-specific primers: forward: 5′-CCCACCGTCGTATTCGTGAG-3′ (SEQ ID NO:9) and reverse: 5′-TCAGGGCGATGGTTTTGTCCC-3′ (SEQ ID NO:10). Known copy numbers of the corresponding vector plasmids were used for the generation of the standard curves. The AAV9 viral vectors yielded high titers (typically 10¹² to 10¹³ vector genomes (vg)/ml).

Animal Studies

Four to five-weeks old CB17 SCID mice were intravenously injected via the tail vein with 3×10¹⁰ vector genomes (vg) per mouse (2-3 mice were injected per group) with AAV9 viral vectors that expressed the luciferase gene from the CARD-CRE3, -CRE11 or -CRE12/SPc5-12 promoter or a control AAV9 vector devoid of any CRE. An AAV vector encoding the luciferase reporter gene driven from a cytomegalovirus (CMV) promoter was used as a reference for comparison. The AAV9-CMV vector was used as a reference construct because most experiments conducted for cardiovascular diseases use the CMV promoter to drive transgene expression.

To assess the impact of CARD-CRE on gene expression an in vivo screening platform was used based on assessing luciferase reporter activity using bioluminescence imaging (BLI) or luminometric analysis (FIG. 2). Luciferase expression was determined in the heart, skeletal muscle (i.e. quadriceps, gastrocnemius, tibialis, triceps, biceps, diaphragm) and non-muscle tissues such as liver, kidney, spleen, lung and brain. Luminometric analysis was performed on purified cardiomyocytes.

Dissociation of Adult Mouse Heart and Isolation and Purification of Cardiomyocytes
1. The adult mice were anaesthetized with isoflurane in a closed chamber.
2. The chest was cut open to expose the heart. 3. The descending aorta and vena cava were cut and 7 ml of EDTA buffer (130 mM NaCl, 5 mM KCl, 0.5 mM NaH₂PO₄, 10 mM HEPES, 10 mM glucose, 10 mM 2,3-butanedione monoxime (BDM), 10 mM taurine, 5 mM EDTA. pH 7.8) was injected into the right ventricle immediately to flush out blood.
4. The heart was clamped at the ascending aorta, which was then cut to resect the heart.
5. The heart was then transferred to a 15 ml falcon containing fresh EDTA buffer till the tissue was completely submerged until further processing.
6. The heart was transferred to a 60 mm petri dish and attached to the syringe pump infusion system (SP220IZ) via the left ventricle (the lower bulged part of the heart) using a 25 G butterfly needle.
7. The heart was perfused with 10 ml EDTA buffer at a rate of 2 ml per minute.
8. The heart was then transferred to a new dish containing perfusion buffer (130 mM NaCl, 5 mM KCl, 0.5 mM NaH₂PO₄, 10 mM HEPES, 10 mM glucose, 10 mM BDM, 10 mM taurine, 1 mM MgCl₂. pH 7.8).
9. 7 ml of perfusion buffer was injected in a similar way into the left ventricle via the same perforation at a rate of 2 ml per minute.
10. Then the heart was transferred to a new dish containing warm (37° C.) collagenase buffer (0.5 mg/mL collagenase 2 (Thermo Fisher Scientific, Catalogue No.: 17101015), 0.5 mg/mL collagenase 4 (Thermo Fisher Scientific, Catalogue No.: 17104019), 0.05 mg/mL protease XIV (Sigma. Cat. No. P5147) was dissolved in perfusion buffer.).
11. Sequentially 10 ml of warm collagenase buffer was injected 4 times into the left ventricle at a rate of 2 ml per minute.
12. The heart was transferred into a new 60 mm petri dish containing 3 ml of collagenase buffer.
13. The tissue was teased gently into 1 mm×1 mm sized pieces with forceps.
14. The tissue was then triturated gently for 2 minutes using a 1000 μl pipette. The tip of the 1000 μl pipette was cut at the end of it to increase the pore size of the tip and ease out the trituration process.
15. The enzymatic digestion was stopped by adding the cell suspension to 5 ml warm (37° C.) stop solution (perfusion buffer containing 5% sterile fetal bovine serum (FBS).) in a petri dish on a rotary bench.
16. The obtained cell suspension was passed through a 100 um pore size cell strainer into a 50 ml Falcon tube in order to remove the cell debris.
17. The filter was further washed with 5 ml of stop buffer.
18. The cells were allowed to settle by gravity for 20 minutes. Half of the supernatant was transferred into a new 50 ml falcon tube for endothelial cell isolation.
19. The cell suspension with the pellet was further subjected to gravity settling in 15 ml falcons two more times with 4-5 ml perfusion buffer.
20. The cell pellet formed was resuspended in 2 ml perfusion buffer and a cell count of the formed myocyte population was taken using a haemocytometer.

Analysis of Luciferase Activity in the Isolated Cells:
1. After cardiomyocytes isolation, the total cell number was counted using a haemocytometer.
2. The required cell suspension was taken in separate 1.5 ml or 2 ml eppendorf tube.
3. The the cell suspension was spin in eppendorf tube in a tabletop centrifuge at 8000-9000 rpm for 2 min.
4. The supernatant was discarded and the cell pellet was taken for analysis according to the ONE-Glo™ EX Luciferase Assay System according to the manufacturer's instructions.
5. 410 μl of ONE-Gb™ EX Reagent was added to the cell pellet.
6. The cell pellet and ONE-Gb™ EX Reagent were mixed few times by pipetting for cell lysis and thereafter incubated for 3 min at room temperature.
7. Then 410 μl of mixture was divided equally into 2 adjacent wells of a 96 well plate (200 μl/well) (Greiner Bio, Cat #655073)
8. After adding the mixture, the 96 well plate was placed into the plate holder of the GloMax Explorer system. The plate was inserted as per the instruction displayed on the machine.

Results

In Vivo Screening of Cardiomyocyte-Derived Cis Regulatory Elements (CARD-CRE) by Bioluminescence Imaging (BLI)

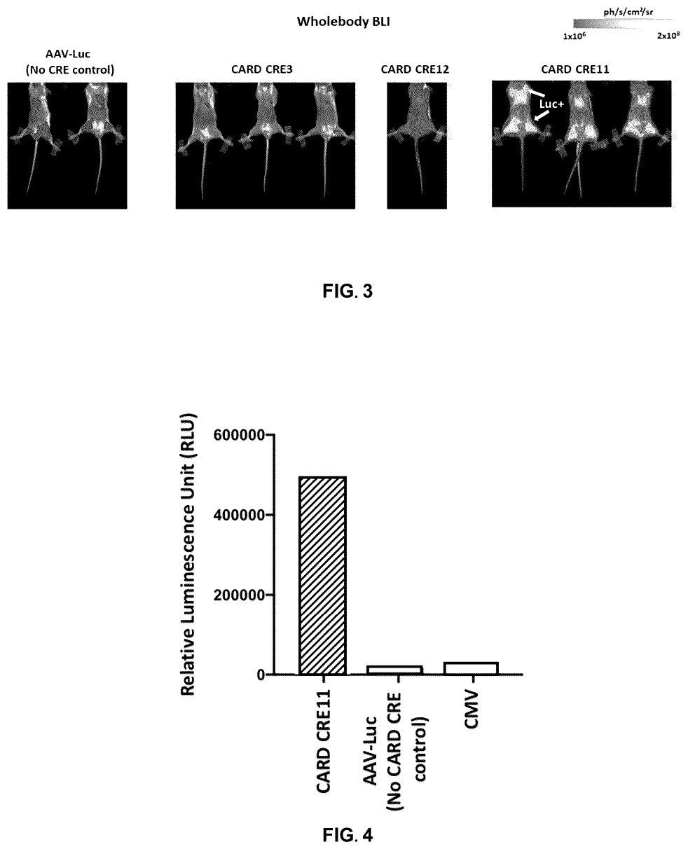

Four to five-weeks old CB17 SCID mice were intravenously injected via the tail vein with 3×10¹⁰ vector genomes (vg) per mouse (2-3 mice were injected per group) with AAV9 vectors that expressed the luciferase gene from the SPc5-12 promoter operably linked to CARD-CRE3, -CRE11 or -CRE12 regulatory elements identified in Example 1, or a control AAV9 vector devoid of any CRE. The result of the whole body BLI are shown in FIG. 3.

Whole body BLI (FIG. 3) clearly showed that the highest level of luciferase activity (white area) was observed in mice injected with the AAV vector containing the CARD-CRE11 regulatory element as compared to the control AAV devoid of any CARD-CRE. A robust enhancement of luciferase activity was apparent. CARD-CRE12 led to a relatively modest enhancement of luciferase activity whereas CARD-CRE3 failed to increase luciferase activity. Hence, a relatively robust increase in gene expression was seen with CARD-CRE11.

In Vivo Validation of the Cardiomyocytes Derived Cis Regulatory Elements (CARD-CRE) by Luminometric Analysis

Mice were injected with AAV9-CARD-CRE11 vector (n=2) or a control AAV9 vector (n=2) devoid of any CRE. Mice injected with an AAV vector encoding the luciferase reporter gene driven from a cytomegalovirus (CMV) promoter was used as a reference for comparison (n=2). About two weeks post vector injection, mice were sacrificed and the heart of each mouse was isolated. Cardiomyocytes were subsequently purified per protocol. Freshly purified cardiomyocytes from the 3 different groups of mice were counted and 50.000 cardiomyocytes were taken for measuring luciferase activity using a luminometer. Luciferase activity is expressed as relative luminescence units (RLU) (FIG. 4).

The results show that the highest level of luciferase expression was observed from mice injected with the AAV vector containing the CARD-CRE11 regulatory element compared to the control AAV vector devoid of any CARD-CRE. Up to a 30-fold difference in luciferase activity was apparent. Moreover, the AAV9 vector containing the CARD-CRE11 regulatory element yielded a 20-fold increase in luciferase activity compared to the CMV reference vector. This demonstrates that CARD-CRE11 can lead to an unexpected robust increase in gene expression in cardiomyocytes, which is advantageous for cardiovascular gene therapy.

Conclusions

CARD-CRE11 led to robust increase in gene expression in the heart and other muscle tissues, which is advantageous for the treatment of cardiovascular and muscle related diseases by gene therapy.

### Example 3: In Vivo Validation of the Identified CARD-CRE11 Regulatory Element

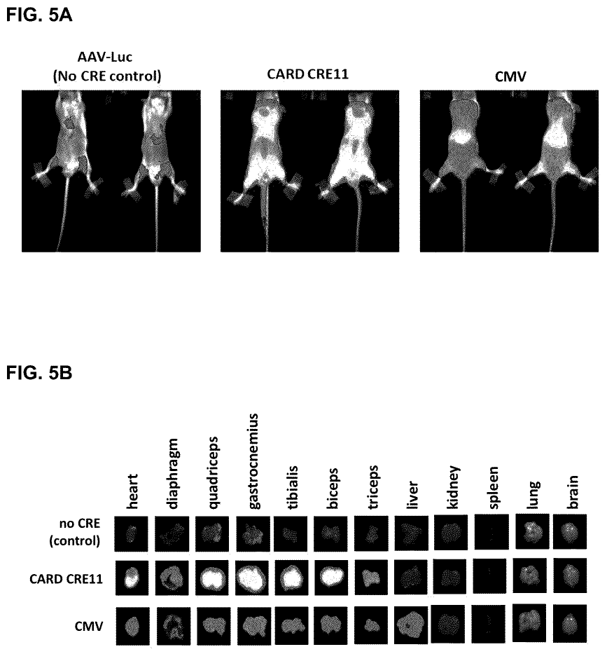

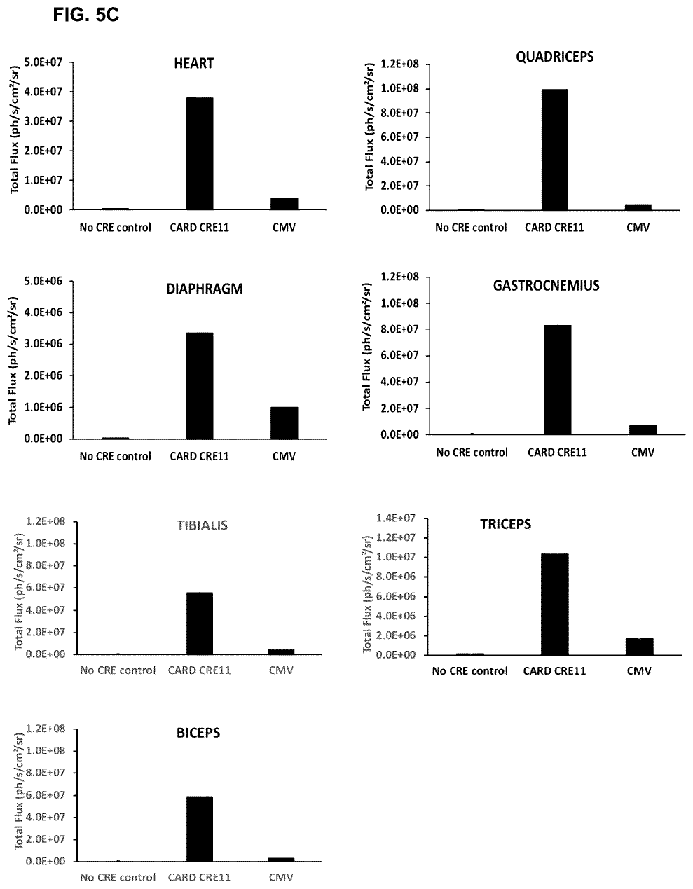

To confirm the robustness of the CARD-CRE11 regulatory element as observed in Example 2, a separate experiment was performed as described in Example 3 using four to five weeks old CB17 SCID mice which were intravenously injected via tail vein with 3×10¹⁰ vg per mouse (n=2) of AAV9-CARD-CRE11, a control AAV vector (n=2) devoid of any CRE (no CRE control) to assess the impact of CARD-CRE11 on gene expression in the heart and other organs, and the AAV9-CMV reference vector (n=2). The result of the whole body BLI as well as 12 different individual organ/tissues are shown in FIGS. 5A and 5B. Quantification of the BLI analysis is shown in FIG. 5C.

Whole-body BLI (FIG. 5A) showed that the highest level of luciferase activity (white area) was observed from mice injected with the AAV vector containing the CARD-CRE11 regulatory element as compared to the control AAV devoid of any CARD-CRE. Moreover, the inclusion of the CARD-CRE11 regulatory element led to robust increase of gene expression, which was higher than when the reference CMV promoter was used to drive luciferase expression.

The results were consistent with the luciferase activity obtained based on isolated organs and tissues retrieved upon dissection of the injected animals (FIG. 5B). The AAV vector containing the CARD-CRE11 regulatory element showed higher levels of luciferase activity in the heart and all the muscle tissues (i.e. diaphragm, quadriceps, gastrocnemius, tibialis, triceps and biceps) compared to the heart and muscle tissues obtained from mice injected with the control AAV9 vector devoid of any CARD-CRE or compared to mice injected with the CMV reference vector. In contrast, the CMV reference vector also yielded luciferase expression in non-muscle tissues, particularly the liver (FIG. 5B & 5C).

Quantification of luciferase activity in the individual organs and tissues showed that inclusion of the CARD-CRE11 regulatory element increased luciferase expression −70-fold in the heart and −80-130 fold in the various skeletal muscle groups (i.e. diaphragm, quadriceps, gastrocnemius, tibialis, triceps and biceps) compared to the control vector devoid of any CARD-CRE. Inclusion of the CARD-CRE11 regulatory element increased luciferase expression −10 fold in the heart and −3-20 fold in the various skeletal muscle groups (i.e. diaphragm, quadriceps, gastrocnemius, tibialis, triceps and biceps) compared to the reference vector with the CMV promoter.

Conclusions

The data shown in FIG. 5 clearly confirms that CARD-CRE11 led to robust increase in gene expression in the heart and other muscle tissues and yielded significantly higher expression compared to the reference CMV vector, which is advantageous for the treatment of cardiovascular and muscle-related diseases by gene therapy.

### Example 4: In Vivo Validation of Identified Cardiomyocyte-Derived Nucleic Acid Regulatory Elements (CARD-CREs)

Several CARD-CREs identified in Example 1, in particular CARD-CRE14, CARD-CRE16 and CARD-CRE17, were compared side by side for their ability to increase gene expression as described in Example 2.

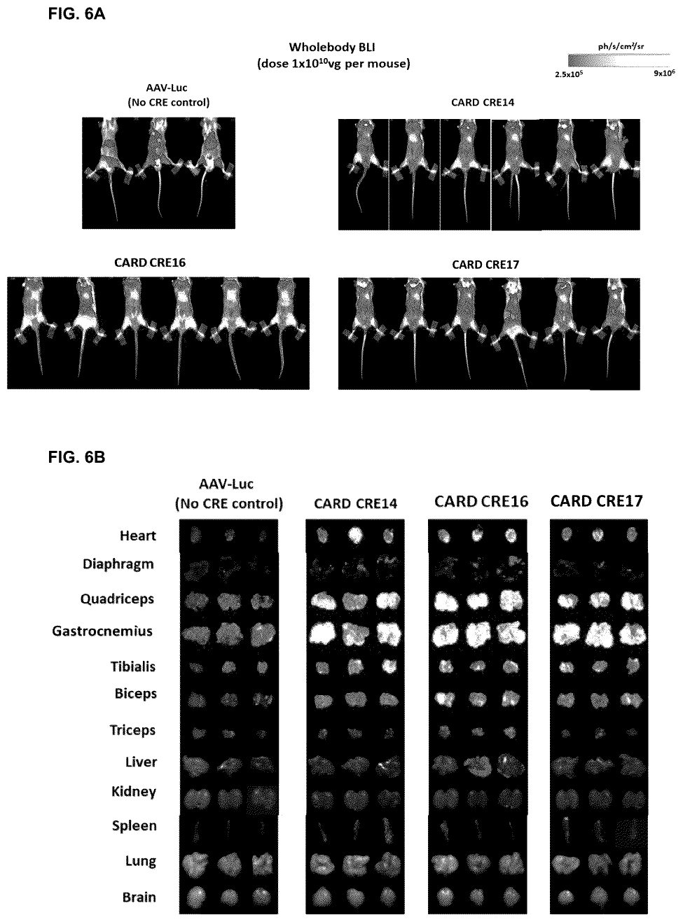

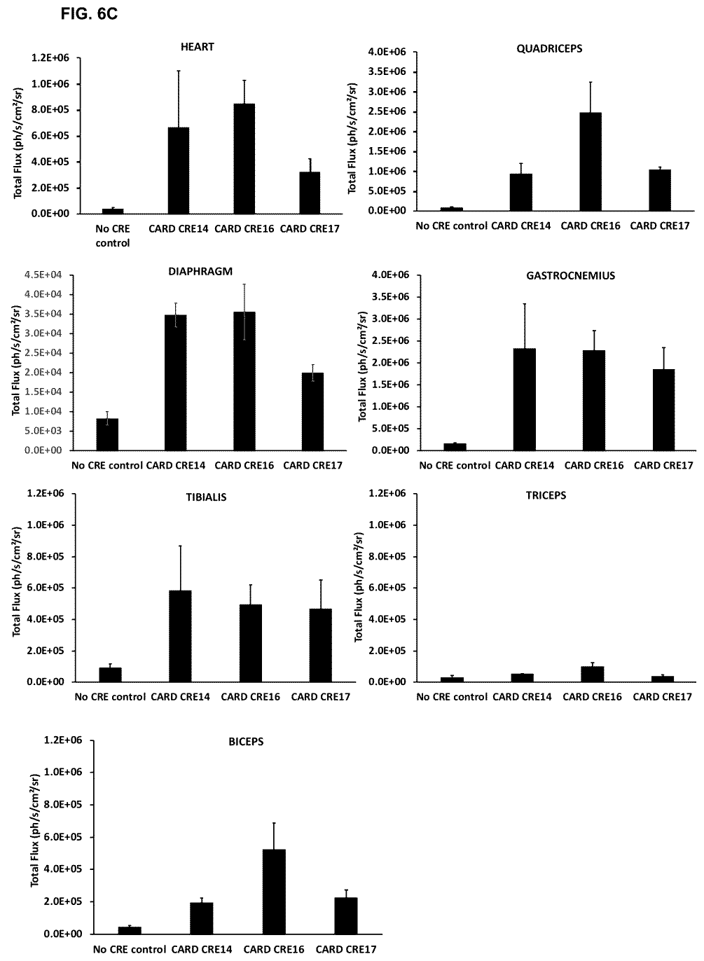

In this experiment, 4-5 weeks old CB17 SCID mice were intravenously injected via the tail vein with 1×10¹¹ vg per mouse (3 mice were injected per group) of AAV9 vectors carrying the luciferase gene driven from the SPc5-12 promoter operably linked to a CARD-CRE regulatory element or a control AAV9 vector devoid of any CRE (no CRE control) to assess the impact of the selected CARD-CRE on gene expression in the heart, the muscle and other organs. The result of the whole body BLI is shown in FIG. 6A, and the results in 12 different individual organ/tissues retrieved upon dissection of the injected animals are shown in FIGS. 6B & 6C.

Whole-body BLI (FIG. 6A) showed that luciferase activity (white area) was increased in mice injected with the AAV vector containing the different CARD-CREs (i.e. CARD-CRE14, CARD-CRE16 and CARD-CRE17) as compared to the control AAV vector devoid of an CARD-CRE. The results were consistent with the luciferase activity obtained based on isolated organs and tissues retrieved upon dissection of the injected animals (FIG. 6B). The AAV vector containing the CARD-CRE14, CARD-CRE16 and CARD-CRE17 showed higher levels of luciferase activity in the heart and all the muscle tissues (i.e. diaphragm, quadriceps, gastrocnemius, tibialis, triceps and biceps) compared to the heart and muscle tissues obtained from mice injected with the control AAV9 vector devoid of a CARD-CRE. Barely any luciferase activity was detected in non-muscle tissues such as the liver, kidney, spleen, lung and brain.

Quantification of luciferase activity in the individual organs and tissues showed that inclusion of CARD-CRE14, CARD-CRE16 and CARD-CRE17 increased luciferase expression in the heart and in most of the various skeletal muscle groups compared to the control vector devoid of a CARD-CRE (FIG. 6C). In particular, in the heart CARD-CRE14, CARD-CRE16 and CARD-CRE17 increased luciferase activity 18-, 23- and 9-fold, respectively when compared to the control vector devoid of a CARD-CRE. CARD-CRE14 increased luciferase activity in the gastrocnemius (16-fold), quadriceps (11-fold), tibialis (6-fold), biceps (4-fold), diaphragm (4-fold) and to a lesser extent in the triceps (1.6-fold) compared to the control vector. Similarly, CARD-CRE16 increased luciferase activity in the gastrocnemius (16-fold), quadriceps (30-fold), tibialis (5-fold), biceps (12-fold), diaphragm (4-fold) and triceps (3-fold) compared to the control vector. CARD-CRE17, also increased luciferase activity in the gastrocnemius (13-fold), quadriceps (12-fold), tibialis (5-fold), biceps (5-fold), diaphragm (2.5-fold) and only marginally in triceps (1.3-fold) compared to the control vector. The increased luciferase activity in the heart and various muscle groups by CARD-CRE14, CARD-CRE16 and CARD-CRE17 compared to the control vector is summarized in Table 2.

TABLE 2
Overview of increased luciferase activity by CARD-CRE14, CARD-CRE16 and CARD-CRE17.

| | Fold increased in luciferase expression in different organs as compared to control vector | | | | | | |
|---|---|---|---|---|---|---|---|
| | without CARD CRE | | | | | | |
| CARD CRE: | Heart | Gastrocnemius | Diaphragm | Quadriceps | Tibialis | Triceps | Biceps |
| CARD CRE 14 | 18 | 16 | 4 | 11 | 6 | 1.6 | 4 |
| CARD CRE 16 | 23 | 16 | 4 | 30 | 5 | 3 | 12 |
| CARD CRE 17 | 9 | 13 | 2.5 | 12 | 5 | 1.3 | 5 |

Conclusions

The data shown in FIG. 6 demonstrates that CARD-CRE14, CARD-CRE16 and CARD-CRE17 led to a robust increase in gene expression in the heart and other muscle tissues, which is advantageous for the treatment of cardiovascular and muscle-related diseases by gene therapy.

### Example 5: In Vivo Validation of Identified Cardiomyocyte-Derived Nucleic Acid Regulatory Elements (CARD-CREs)

Several CARD-CREs identified in Example 1, in particular CARD-CRE8, CARD-CRE11, CARD-CRE16 and CARD-CRE20, were compared side by side for their ability to increase gene expression as described in Example 2.

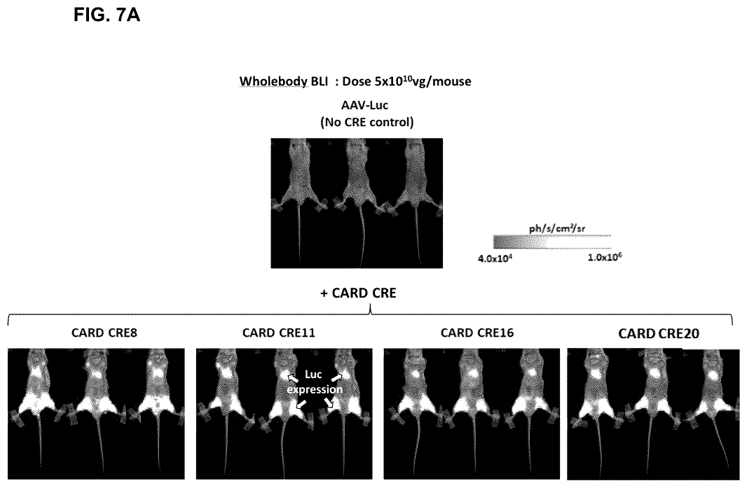

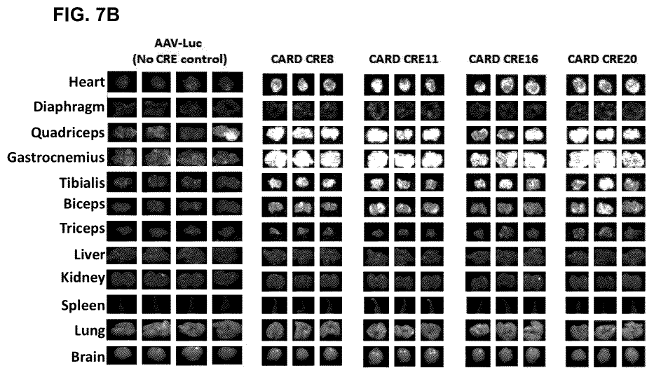

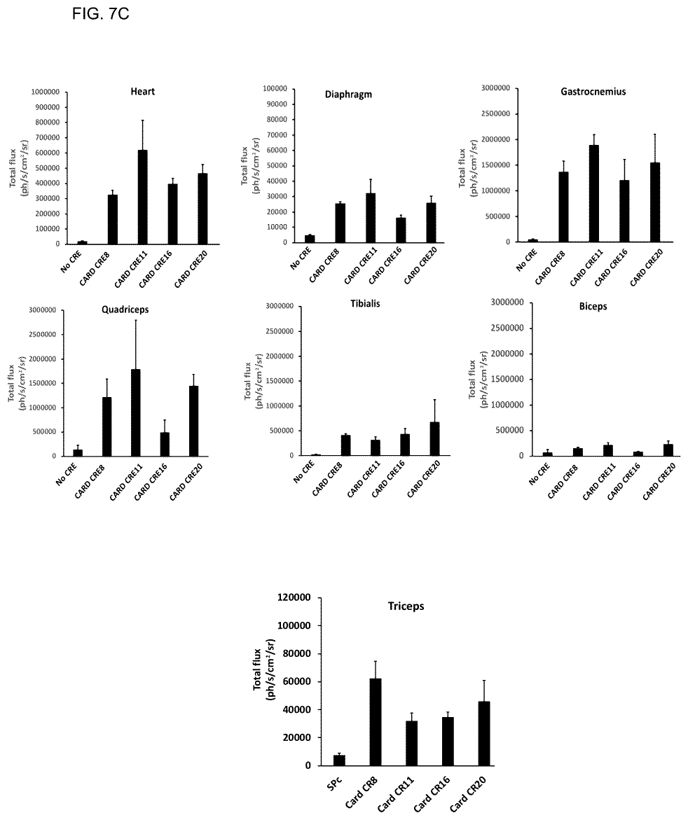

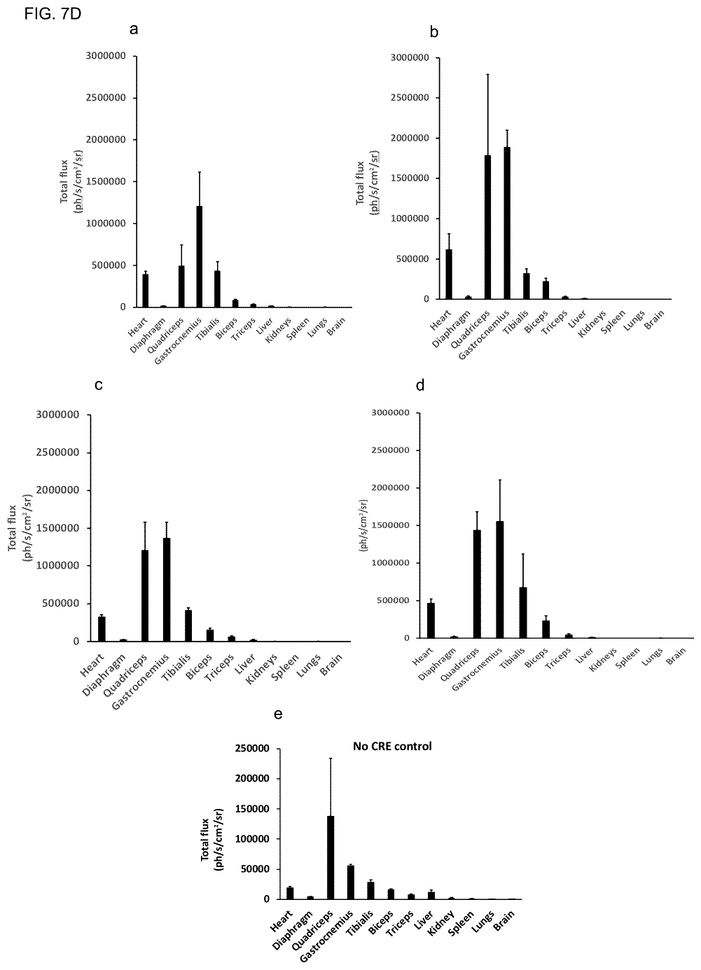

In this experiment, 4-5 weeks old CB17 SCID mice were intravenously injected by tail vein with 5×10¹⁰ vg per mouse (3 mice were injected per group). Control AAV9 vector devoid of any CRE (no CRE control) was used for comparison to assess the impact of a given CARD-CRE on gene expression in the heart and other muscle groups and organs. The result of the whole body BLI as well as 12 different individual organ/tissues retrieved upon dissection of the injected animals are shown in FIG. 7.

Whole-body BLI (FIG. 7A) showed that the luciferase activity (white area) was increased in all the vectors containing the CARD-CREs (i.e. CARD-CRE8, CARD-CRE11, CARD-CRE16 and CARD-CRE20) when compared to the AAV control vector devoid of any CARD-CRE.

Similar results were observed when 12 individual tissues and organs were quantified by BLI for the luciferase expression (FIG. 7B, 7D). In particular, the CARD-CRE8, CARD-CRE11, CARD-CRE16 and CARD-CRE20 increased luciferase activity in the heart and all the muscle tissues tested (i.e. diaphragm, quadriceps, gastrocnemius, tibialis, triceps and biceps). Barely any expression was detected in non-muscle tissues such as the liver, kidney, spleen, lung and brain.

The four different selected CARD-CREs (i.e. CARD-CRE8, CARD-CRE11, CARD-CRE16 and CARD-CRE20) increased luciferase activity 17 to 32-fold compared to the control vector without CARD-CRE in the heart (FIG. 7C). Similarly, CARD-CREs enhanced luciferase activity in all the other different organs tested: diaphragm (3-7 fold), quadriceps (4-13 fold), gastrocnemius (22-34 fold), tibialis (12-24 fold), triceps (4-8 fold) and biceps (1-3 fold) (FIG. 7C, Table 3).

TABLE 3
Overview of increased luciferase activity by CARD-
CRE8, CARD-CRE11, CARD-CRE16 and CARD-CRE20.

| | Fold increased in luciferase expression in different organs as compared to control vector | | | | | | |
|---|---|---|---|---|---|---|---|
| | without CARD CRE | | | | | | |
| CARD CRE: | Heart | Gastrocnemius | Diaphragm | Quadriceps | Tibialis | Triceps | Biceps |
| CARD CRE 8 | 17 | 25 | 5 | 9 | 15 | 8 | 2 |
| CARD CRE 11 | 32 | 34 | 7 | 13 | 12 | 4 | 3 |
| CARD CRE 16 | 21 | 22 | 3 | 4 | 16 | 5 | 1 |
| CARD CRE 20 | 24 | 28 | 6 | 10 | 24 | 6 | 3 |

Conclusion

The data shown in FIG. 7 demonstrate that CARD-CRE8, CARD-CRE11, CARD-CRE16 and CARD-CRE20 led to a robust increase in gene expression in the heart and other muscle tissues, which is advantageous for the treatment of cardiovascular and muscle-related diseases by gene therapy.

General Conclusion

Based on the data shown in Examples 2-5, the most robust increase of transgene expression was observed from the following CARD-CREs: CARD-CRE8 (SEQ ID NO. 2), CARD-CRE11 (SEQ ID NO.3), CARD-CRE14 (SEQ ID NO.5), CARD-CRE16 (SEQ ID NO.6), CARD-CRE17 (SEQ ID NO.7) and CARD-CRE20 (SEQ ID NO.8). These CARD-CREs were identified by data mining of RNA-seq expression data, derived from the top highly expressed genes in normal human cardiomyocytes (Example 1).

### Example 6: In Vivo Validation of the Identified CARD-CRE11 Regulatory Element Operably Linked to the hMLC Promoter

Materials & Methods

Generation of AAV Vectors

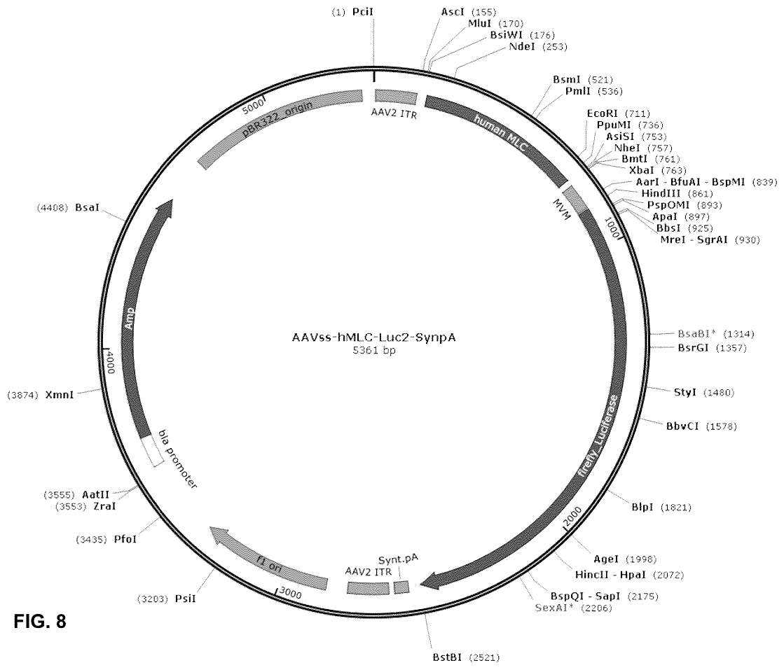

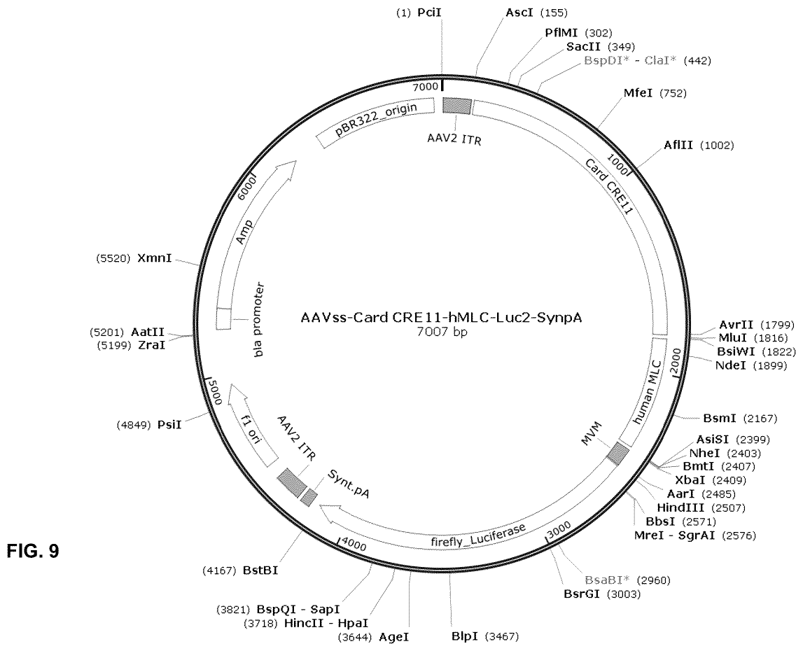

The human myosin light chain (hMLC) promoter (567 bp, SEQ ID NO:14) was synthesized by conventional DNA synthesis and cloned into a single stranded AAV vector backbone as described in Example 2 without or with the CARD-CRE11 element identified in Example 1 to generate the AAVss-hMLC-Luc2-SynpA vector (FIG. 8) or AAVss-Card CRE11-hMLC-Luc2-SynpA vector (FIG. 9), respectively, wherein “Luc2” refers to the luciferase reporter, “SynpA” refers to synthetic polyadenylation site, AAVss refers to the single-stranded vector configuration and AAV9 refers to AAV serotype 9.

AAV Production

The corresponding AAV9 vector particles were manufactured by Signagen and vector titer was determined as described in Example 2.

Bioluminescence Imaging Analysis

Adult CB17/IcrTac/Prkdcscid (alias CB17 SCID) male mice (5-6 weeks old; weight: 18-21 g) were injected with the AAV9 vectors at a dose corresponding to 10¹¹ vg per mouse. Mice were euthanized 4 weeks post vector injection and the luciferase activity was subsequently determined by bioluminescence imaging in the dissected heart compared to dissected individual muscle types. A hand-drawn region of interest (ROI) was used for every individual tissue. Luciferase expression from the individual tissue was measured as total flux, expressed in photons/sec/cm²/sr (mean+s.e.m.; n=3)

Results

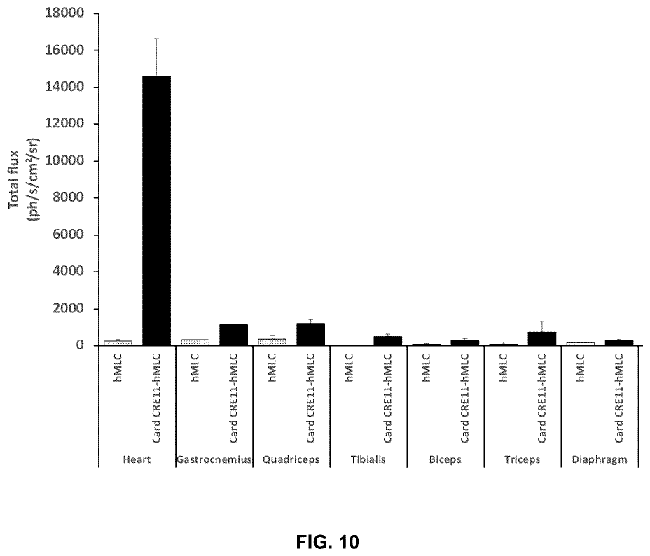

CARD-CRE11 selectively enhanced luciferase activity from the hMLC promoter most strongly in the heart (up to 30-fold) compared to any other skeletal muscle or diaphragm tissue (FIG. 10).

### Example 7: In Vivo Validation of the Identified CARD-CRE11 Regulatory Element Operably Linked to the Ubiquitously Expressed CMV Promoter

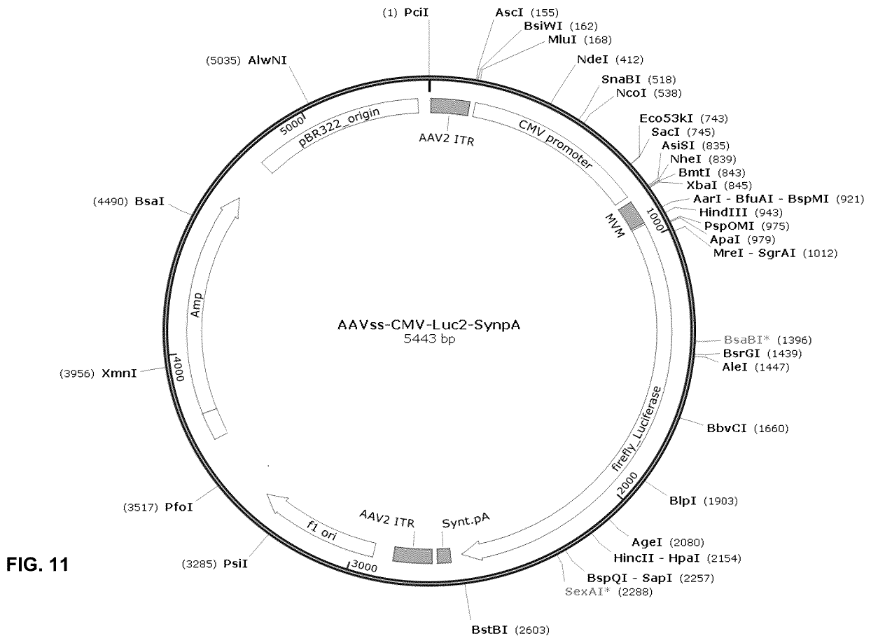

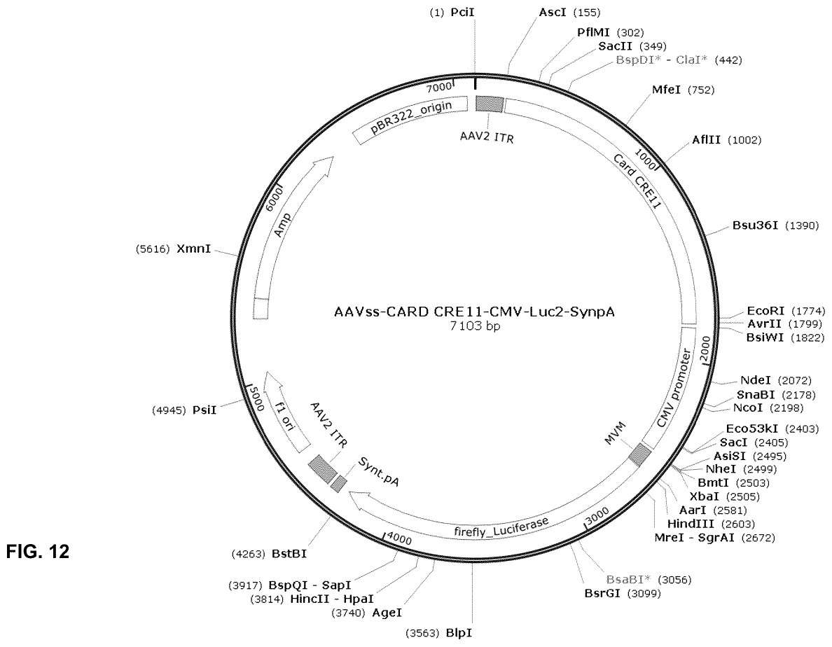

Materials and methods were used/applied as described in Example 6, wherein the CARD-CRE11 element was cloned upstream of the CMV promoter in the AAVss-CMV-Luc2-SynpA vector (FIG. 11) to generate the AAVss-CARD-CRE11-CMV-Luc2-SynpA vector (FIG. 12).

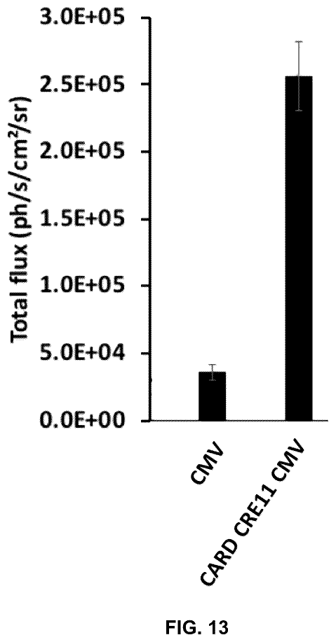

CARD-CRE11 also increased luciferase activity (7-fold) from the ubiquitously expressed promoter CMV in the heart compared to a control CMV-luciferase vector devoid of CARD-CRE11 (FIG. 13).

1. The nucleic acid expression cassette of claim 3, wherein the nucleic acid regulatory element has a maximal length of 2000 nucleotides.

2. The nucleic acid expression cassette of claim 3, wherein the nucleic acid regulatory element enhances heart- and/or muscle-specific gene expression.

3. A nucleic acid expression cassette comprising at least one nucleic acid regulatory element operably linked to a promoter and a transgene, wherein said nucleic acid regulatory element comprises a sequence selected from the group consisting of: SEQ ID NO:3, SEQ ID NO:2, SEQ ID NO:5, SEQ ID NO:6, SEQ ID NO:7, SEQ ID NO:8, a sequence having at least 80% identity to the full-length of any of these sequences, and functional fragments thereof.

4. The nucleic acid expression cassette according to claim 3, wherein the promoter is a heart- and/or muscle-targeted promoter.

5. The nucleic acid expression cassette of claim 3, wherein the promoter is a ubiquitously expressed promoter.

6. The nucleic acid expression cassette of claim 3, wherein the transgene encodes a therapeutic protein or an immunogenic protein.

7. The nucleic acid expression cassette of claim 3, wherein the transgene encodes a non-coding RNA.

8. The nucleic acid expression cassette of claim 3, wherein the nucleic acid expression cassette further comprises an intron.

9. The nucleic acid expression cassette of claim 3, wherein the nucleic acid expression cassette further comprises a polyadenylation signal.

10. The nucleic acid expression cassette of claim 3, wherein the nucleic acid expression cassette is comprised in a vector.

11. The nucleic acid expression cassette of claim 10, wherein the vector is a viral vector.

12. The nucleic acid expression cassette of claim 3, wherein the nucleic acid expression cassette is comprised in a pharmaceutical composition with a pharmaceutically acceptable carrier.

13. (canceled)

14. (canceled)

15. (canceled)

16. A method, for expressing a transgene product in heart and/or muscle cells, comprising:
introducing the nucleic acid expression cassette of claim 3 into the heart and/or muscle cells; and
expressing the transgene product in the heart and/or muscle cells.

17. The nucleic acid expression cassette of claim 3, wherein the nucleic acid regulatory element comprises a sequence having at least 95% sequence identity to the full length of a sequence selected from the group consisting of: SEQ ID NO:3, SEQ ID NO:2, SEQ ID NO:5, SEQ ID NO:6, SEQ ID NO:7, SEQ ID NO:8, and functional fragments thereof.

18. The nucleic acid expression cassette of claim 3, wherein the promoter is selected from the group consisting of the hMLC promoter, the SPc5-12 promoter, the desmin (DES) promoter, and the MTICK7 promoter.

19. The nucleic acid expression cassette of claim 3, wherein the promoter is a CMV promoter, a RNA polymerase II promoter, or a RNA polymerase III promoter.

20. The nucleic acid expression cassette of claim 7, wherein the non-coding RNA is selected from the group comprising a microRNA, a long non-coding RNA, a circular RNA, and a small interfering RNA

21. The method according to claim 16, wherein the heart and/or muscle cells are cardiomyocytes.

22. the method according to claim 16, further comprising recovering the transgene product from the heart and/or muscle cells or culture medium.
